(19) United States
(12) Patent Application Publication
(10) Pub. No.: US 2009/0306181 A1
(43) Pub. Date: Dec. 10, 2009

(54) Compositions and methods for evaluating and treating heart failure

(75) Inventors: Sadakatsu Ikeda, New York, NY (US); William T. Pu, Chestnut Hill, MA (US); Sek Won Kong, Arlington, MA (US)

Correspondence Address: WOLF GREENFIELD & SACKS, P.C., 600 ATLANTIC AVENUE, BOSTON, MA, 02210-2206, US

(73) Assignee: Children's Medical Center Corporation, Boston, MA (US)

(21) Appl. No.: 12/311,456

(22) PCT Filed: Sep. 28, 2007
(86) PCT No.: PCT/US07/20883

§ 371 Date: Jun. 22, 2009

Related U.S. Application Data
(60) Provisional application No. 60/848,212, filed on Sep. 29, 2006.
(60) Provisional application No. 60/965,699, filed on Aug. 21, 2007.

(51) Int. Cl. A61K 31/7052; C12Q 1/68; C12N 5/00; A61P 9/00

(52) U.S. Cl. 514/44 A; 435/6; 435/375

(57) ABSTRACT

The invention relates to compositions, formulations, kits, and methods useful for the treatment and evaluation of heart disease in an individual.

### RELATED APPLICATIONS

This application is related to U.S. Provisional Application U.S. Ser. No. 60/848,212 (Attorney Docket No.: 104778.00006) filed Sep. 29, 2006 and U.S. Provisional Application U.S. Ser. No. 60/965,699 (Attorney Docket No.: C1233.70003US01) filed Aug. 21, 2007. The entire teachings of the referenced provisional applications are expressly incorporated herein by reference.

### GOVERNMENT FUNDING

This application was made with government support under Grant No. HL66582, awarded by the National Institutes of Health. The government has certain rights in the invention.

### TECHNICAL FIELD

The invention relates to compositions, formulations, kits, and methods useful for the treatment and evaluation of heart disease in an individual.

### BACKGROUND OF THE INVENTION

Heart disease encompasses a family of disorders, such as cardiomyopathies, and is a leading cause of morbidity and mortality in the industrialized world. Disorders within the heart disease spectrum are understood to arise from pathogenic changes in distinct cell types, such as cardiomyocytes, via alterations in a complex set of biochemical pathways. For example, certain pathological changes linked with heart disease can be accounted for by alterations in cardiomyocyte gene expression that lead to cardiomyocyte hypertrophy and impaired cardiomyocyte survival and contraction. Thus, an ongoing challenge in the development of heart disease treatments has been to identify specific therapies for each particular heart disease. Achieving this goal requires advances in both heart disease classification and the development of targeted therapeutic modalities.

### SUMMARY OF THE INVENTION

An ongoing challenge of heart disease treatment has been to target specific therapies to particular heart disease types in a manner that maximizes effectiveness and minimizes toxicity. Improvements in heart disease classification and therapeutic modalities have thus been central to advances in heart disease treatment. Described herein are methods useful for the evaluation of heart disease based on the levels or occurrence of microRNA expression. For example, in one embodiment, the method comprises assessing the occurrence or level of a (at least one) microRNA or assessing microRNA expression patterns in a heart tissue sample and based on the results of that assessment, assigning the heart tissue sample (e.g., a myocardium sample) to a known or putative heart disease class such as ischemic cardiomyopathy, dilated cardiomyopathy, or aortic stenosis. Also described herein is a method of predicting the response of an individual to treatment of (to a therapeutic regime for) heart disease, based on microRNA expression patterns in an individual in need thereof. The present invention also relates to methods, formulations, and kits that are useful for the treatment of heart disease and that are based on microRNAs associated with heart disease. For example, one embodiment involves the use of small-interfering nucleic acids to supplement or inhibit microRNAs associated with heart disease. In some embodiments, the supplementation or inhibition of microRNAs comprises contacting a myocardial cell with a small-interfering nucleic acid that is identical to, or complementary to, a microRNA associated with heart disease. As used herein, the term myocardial cell includes any cell that is obtained from, or present in, myocardium such as a human myocardium and/or any cell that is associated, physically and/or functionally, with myocardium. In one embodiment, a myocardial cell is a cardiomyocyte. In some embodiments, the supplementation or inhibition of microRNAs comprises contacting a myocardial cell with a small-interfering nucleic acid that is substantially similar to, or substantially complementary to, a microRNA associated with heart disease. Described herein are methods for determining or identifying microRNAs useful for classification of samples obtained from individuals, methods for determining the importance of a microRNA involved in heart disease, and treatment strategies for heart disease based on modulating microRNA activity in myocardial cells.

In one embodiment, the invention relates to methods for assessing the risk of heart disease, or aiding in assessing the risk of heart disease, in an individual in need thereof, comprising determining the occurrence or level of a (at least one, one or more) microRNA in the myocardium (e.g., in myocardial tissue, mycocardial cells or myocardial cell components, such as DNA or RNA) of the individual, wherein if the occurrence or level of the microRNA in the myocardium (e.g., in myocardial tissue, mycocardial cells or myocardial cell components, such as DNA or RNA) of the individual is different from the occurrence or level of the microRNA in the myocardium (e.g., in myocardial tissue, mycocardial cells or myocardial cell components, such as DNA or RNA) of a control individual who does not have heart disease, the individual is at risk of having heart disease.

In one embodiment, the invention relates to methods for diagnosing, or aiding in diagnosing, heart disease in an individual in need thereof, comprising determining the occurrence or level of a microRNA in the myocardium (e.g., in myocardial tissue, mycocardial cells or myocardial cell components, such as RNA) of the individual, wherein a difference in the occurrence or level of the microRNA in the myocardium (e.g., in myocardial tissue, mycocardial cells or myocardial cell components, such as RNA) of the individual from the occurrence or level of the microRNA in the myocardium (e.g., in myocardial tissue, mycocardial cells or myocardial cell components, such as RNA) of a control individual who does not have heart disease, is indicative of (indicates that) the individual has heart disease.

In some embodiments of the foregoing methods, the heart disease is heart failure (e.g., congestive heart failure), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

In one embodiment, the invention relates to a method of assessing efficacy of a treatment for heart disease, in an individual in need thereof, wherein the method comprises: (a) determining the occurrence or level of a microRNA in a myocardium sample of the individual before treatment, (b) determining the occurrence or level of the microRNA in a myocardium sample of the individual after treatment, (c) comparing the results of (a) with the results of (b), wherein a difference between the results of (a) and the results of (b) indicates an effect of the treatment. The myocardium sample can be, for example, myocardial tissue, myocardial cells or myocardial cell components, such as RNA. In certain embodiments, the treatment is administration of a drug, such as an ACE inhibitor, an angiotensin II receptor blocker, a Beta-blocker, a vasodilator, a cardiac glycoside, an antiarrhythmic agent, a diuretic, statins, or an anticoagulant, an inotropic agent; an immunosuppressive agent and/or any of the pharmaceutical formulations described herein; use of a pacemaker, defibrillator, mechanical circulatory support; or surgery. In some embodiments, the heart disease is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

In some embodiments of the methods, the microRNA is selected from, or substantially similar to a microRNA selected from, the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p, miR-499, miR-28, miR-222,miR-451, miR422b, let-7g, miR-125a, miR-133a, miR-133b, miR-15a,miR-16, miR-208, miR-30a-5p, miR-30b, miR-30c, miR-30d, miR-335, miR-195, let-7b, miR-27a, miR-27b, let-7c, miR-103, miR-23b, miR-24, miR-342, miR-23a, miR-145, miR-199a*, let-7e, miR-423*, miR-125b, miR-320, miR-93, miR-99b, miR-140*, miR-191, miR-15b, miR-181a, miR-100, and miR-214.

In some embodiments of the methods, the level or occurrence of the microRNA in the myocardium (e.g., in myocardial tissue or myocardial cells) of the individual is less than level of the microRNA in the myocardium (e.g., in myocardial tissue or myocardial cells) of the control individual. In certain embodiments, the microRNA is selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p,miR-499, miR-28, miR-222, miR-451, miR-422b, let-7g, miR-125a, miR-133a, miR-133b, miR-15a, miR-16,miR-208, miR-30a-5p, miR-30b, miR-30c, miR-30d, and miR-335. Similarly, the microRNA can be a microRNA that is substantially similar to one of the aforementioned microRNAs.

In some embodiments of the foregoing methods, the level or occurrence of the microRNA in the myocardium (e.g., in myocardial tissue or myocardial cells) of the individual is greater than level or occurrence of the microRNA in the myocardium (e.g., in myocardial tissue or myocardial cells) of the control individual. In certain embodiments, the microRNA is selected from the group consisting of: miR-195, let-7b, miR-27a, miR-27b,let-7c, miR-103, miR-23b, miR-24, miR-342, miR-23a, miR-145, miR-199a*, let-7e, miR-423*, miR-125b, miR-320, miR-93, miR-99b,miR-140*, miR-191, miR-15b, miR-181a, miR-100, and miR-214. Similarly, the microRNA can be a microRNA that is substantially similar to one of the aforementioned microRNAs.

In one embodiment, the invention relates to a method of determining the type of heart disease in an individual who has heart disease, wherein the method comprises: (a) determining the expression pattern of a set of (e.g., at least one, two or more) microRNAs in a test myocardium sample obtained from the individual; (b) comparing the expression pattern determined in (a) with one or more reference expression patterns, wherein each reference expression pattern is determined from the set of microRNAs in a reference myocardial sample obtained from an individual whose heart disease type is known; (c) categorizing the type of heart disease in the individual as the known heart disease type associated with the reference expression pattern that most closely resembles the expression pattern determined in (a), thereby determining the type of heart disease in the individual who has heart disease. In certain embodiments, each microRNA in the set of microRNAs is selected from the group consisting of: miR-10a, miR-19a,miR-19b,miR-101, miR-30e-5p, miR-126*, miR-374,miR-1,miR-20b,miR-20a, miR-26b, miR-126,miR-106a,miR-17-5p,miR-499,miR-28,miR-222,miR-451, miR-422b, let-7g, miR-125a, miR-133a, miR-133b,miR-15a,miR-16,miR-208, miR-30a-5p, miR-30b, miR-30c,miR-30d, miR-335, miR-195, let-7b, miR-27a, miR-27b, let-7c, miR-103, miR-23b, miR-24, miR-342, miR-23a, miR-145, miR-199a*, let-7e, miR-423*, miR-125b, miR-320, miR-93, miR-99b,miR-140*, miR-191, miR-15b, miR-181a, miR-100, and miR-214. In some embodiments, the known heart disease type is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, or tricuspid regurgitation.

In one embodiment, the invention relates to a method for predicting the response of an individual having heart disease to treatment of the heart disease, wherein the method comprises: (a) determining the expression pattern of a set of microRNAs in a test myocardium sample (e.g., myocardial tissue, myocardial cell) obtained from the individual before the treatment; (b) comparing the expression pattern determined in (a) with one or more reference expression patterns, wherein each reference expression pattern is determined from the set of microRNAs in a reference myocardium sample (e.g., myocardial tissue, myocardial cell) obtained from a control individual having the heart disease, wherein the reference myocardium sample (e.g., myocardial tissue, myocardial cell) was obtained prior to administering, to the control individual, the treatment for the heart disease, and wherein the response of the control individual to the treatment for the heart disease is known; and (c) predicting the response of the individual having heart disease to the treatment for the heart disease as the response to the treatment for the heart disease associated with the control individual having a reference expression pattern that most closely resembles the expression pattern determined in (a), thereby predicting the response of an individual having heart disease to the treatment for the heart disease. In certain embodiments, the treatment is administration of a drug, such as an ACE inhibitor, an angiotensin II receptor blocker, a Beta-blocker, a vasodilator, a cardiac glycoside, an antiarrhythmic agent, a diuretic, statins, or an anticoagulant, an inotropic agent; an immunosuppressive agent and/or any of the pharmaceutical formulations described herein; use of a pacemaker, defibrillator, mechanical circulatory support; or surgery. In some embodiments, the heart disease is heart failure (e.g., congestive heart failure), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

In one embodiment, the invention relates to a method for modulating expression of genes associated with heart disease comprising contacting myocardial cells with an effective amount of a small-interfering nucleic acid capable of inhibiting, in myocardial cells, the expression of a gene product associated with heart disease, wherein the small-interfering nucleic acid comprises a sequence that is substantially similar to, or identical to, the sequence of an miRNA selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p,miR-499, miR-28, miR-222, miR-451, miR-422b, let-7g, miR-125a, miR-133a, miR-133b, miR-15a, miR-16,miR-208, miR-30a-5p, miR-30b, miR-30c, miR-30d, and miR-335. In certain embodiments, the gene product associated with heart disease is CX43, NFAT5, EDN1, CALM1, CALM2, or HDAC4. In some embodiments, the heart disease is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

In one embodiment, the invention relates to a method for reducing calmodulin activity in myocardial cells for the treatment of heart disease, wherein the method comprises contacting myocardial cells with an effective amount of a small-interfering nucleic acid capable of inhibiting CALM1 or CALM2 expression, wherein the small-interfering nucleic acid comprises a sequence that is substantially similar to, or identical to, the sequence of miR-1, thereby reducing calmodulin activity for the treatment of the heart disease. In certain embodiments, the small-interfering nucleic acid comprises the sequence provided in SEQ ID NO: 35. In some embodiments, the heart disease is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

In one embodiment, the invention relates to pharmaceutical formulations useful for modulating expression of genes associated with heart disease, wherein the pharmaceutical formulations comprise: (a) a small-interfering nucleic acid capable of inhibiting, in myocardial cells, the function of a gene product associated with heart disease, wherein the small-interfering nucleic acid comprises a sequence that is substantially similar to, or identical to, the sequence of an miRNA selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p, miR-499, miR-28, miR-222, miR-451, miR-422b, let-7g, miR-125, miR-133a, miR-133b, miR-15a, miR-16, miR-208, miR-30a-5p, miR-30b, miR-30c, miR-30d, and miR-335 and (b) a pharmaceutically acceptable carrier. In one embodiment, the small-interfering nucleic acid comprises the sequence provided in SEQ ID NO: 35. In some embodiments, the heart disease is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation. In some embodiments, a pharmaceutical kit is provided, wherein the kit comprises: any of the forgoing the pharmaceutical formulations and written information (a) indicating that the formulation is useful for inhibiting, in a myocardial cell, the function of a gene associated with the heart disease and/or (b) providing guidance on administration of the pharmaceutical formulation.

In one embodiment, the invention relates to a method for modulating expression of genes associated with heart disease comprising contacting myocardial cells with an effective amount of small-interfering nucleic acid capable of blocking, in myocardial cells, the activity of an miRNA associated with heart disease; wherein the small-interfering nucleic acid comprises a sequence that is substantially complementary to, or complementary to, the sequence of the miRNA associated with heart disease, and wherein the miRNA associated with heart disease is selected from the group consisting of: miR-195, let-7b, miR-27a, miR-27b,let-7c, miR-103, miR-23b, miR-24, miR-342, miR-23a, miR-145, miR-199a*, let-7e, miR-423*, miR-125b, miR-320, miR-93, miR-99b,miR-140*, miR-191, miR-15b, miR-181a, miR-100, and miR-214. In certain embodiments, the small interfering nucleic acid is an antisense oligonucleotide, an antagomir, or an miRNA sponge. In one embodiment, the antisense oligonucleotide is an 2′ O-methyl, locked nucleic acid.

In one embodiment, the invention relates to pharmaceutical formulations useful for modulating expression of genes associated with heart disease, wherein the pharmaceuticals formulations comprise: (a) a small-interfering nucleic acid capable of blocking, in myocardial cells, the activity of an miRNA associated with heart disease; wherein the small-interfering nucleic acid comprises a sequence that is substantially complementary to, or complementary to, the sequence of the miRNA associated with heart disease, and wherein the miRNA associated with heart disease is selected from the group consisting of: miR-195, let-7b, miR-27a, miR-27b,let-7c, miR-103, miR-23b, miR-24, miR-342, miR-23a, miR-145, miR-199*, let-7e, miR-423*, miR-125b, miR-320, miR-93, miR-99b,miR-140*, miR-191, miR-15b, miR-181a, miR-100, and miR-214 and (b) a pharmaceutically acceptable carrier. In some embodiments, the heart disease is heart failure (congestive), ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation. In some embodiments, a pharmaceutical kit is provided, wherein the kit comprises: any of the forgoing the pharmaceutical formulations and written information (a) indicating that the formulation is useful for inhibiting, in myocardial cells, the function of a gene associated with the heart disease and/or (b) providing guidance on administration of the pharmaceutical formulation.

### FIGURES AND DRAWINGS

FIG. 1. Altered miRNA expression in murine and human heart failure. a, Validation of miRNA differential expression by qRTPCR. miRNA level was normalized to U6 expression. n=5 per group. b, Expression of miRNAs in dissociated cardiomyocytes. qRTPCR was used to measure miRNA expression. n=3 for NTg and 7 for CN. *: P<0.05.# P=0.075*: P<0.05 compared with non-failing controls (one way ANOVA with Dunnett's post-hoc test).

FIG. 2. miRNAs broadly influence gene expression. a. mRNA abundance in NTg and MHCα-CN hearts was measured by Affymetrix microarrays. Genes were grouped into four sets: all genes with detectable expression, miR-1 targets, miR-30 targets, and miR-133 targets. Target genes were predicted by TargetScanS. For a given set of genes, the fraction of upregulated genes is the number of upregulated genes divided by the number of genes in the set. Upregulated genes were defined by t-test (P<0.005; n=4 in each group). The likelihood that a randomly selected subset of all genes would yield the fraction of upregulated genes observed among miRNA target sets was calculated by Fisher's exact test. This value is displayed within each bar. b. Cardiomyocyte differentiation in P19CL6 cells is associated with marked upregulation of miR-1, -133, and -208. miR-30b/c showed less dynamic range of expression. Expression was normalized to Gapdh (Gata4 and Nloc2-5) or to U6 (miRNAs) and displayed relative to the level at Day 10, which was defined as 1. c. miR-1, -30b/c, and -133a/b upregulation during P19CL6 differentiation was associated with downregulation of predicted target genes. Affymetrix microarrays were used to measure mRNA level at Day 6 and 10 of P19CL6 differentiation. Downregulated genes were identified by Welch's t-test (P<0.05; n=3). TargetScanS predicted targets of miR-1 and -133 were disproportionately downregulated at a frequency unlikely to occur by chance (numbers within bars, Fisher's exact test).

FIG. 3. Regulation of calmodulin expression by miR-1. a, The 3′UTRs of Calm1 and Calm2 are sufficient to downregulate a reporter in response to miR-1. Sequences to be interrogated for miR-1 responsiveness were cloned downstream of luciferase. These sequences were: reverse complement of miR-1 (miR-1 perfect match; 1 pm); reverse complement of miR-133 (133 pm; negative control); Calm1 3′ UTR; or Calm2 3′ UTR. Reporter activity was measured in the presence of co-transfected miR-1 or unrelated control miRNA (Ctrl). b, miR-1 repression of luciferase reporters requires the miR-1 seed match sequence. Wild-type (WT) reporters contained the 50 bp region encompassing the miR-1 seed match sequence of Calm1 or Calm2. In the mutant (mut) reporter, the miR-1 seed match sequence was mutated at two positions. c. Calmodulin expression in MHCα-CN vs. NTg myocardium. Left panel: Relative mRNA expression of three non-allelic calmodulin-encoding genes was measured by qRTPCR and normalized to Gapdh. Center and right panels: Calmodulin protein level, measured by quantitative western blotting and normalized to Gapdh, was significantly elevated in MHCα-CN myocardium. n=4. d. Calmodulin expression in cultured neonatal rat cardiomyocytes transduced with adenovirus expressing either miR-1 or an unrelated control miRNA. mRNA and protein expression was measured as in c. n=3.*, P<0.05. NS, not significant.

FIG. 4. miR-1 inhibits phenylephrine-induced hypertrophic responses of neonatal rat ventricular cardiomyocytes. Neonatal rat ventricular cardiomyocytes were transduced with adenovirus expressing miR-1 or negative control miRNA (Ctrl). The cells were then stimulated with phenylephrine (20 μM). a. miR-1 inhibited nuclear translocation of NFAT. NFATc3 subcellular localization was determined 24 hours after PE stimulation by immunofluorescent staining. Cardiomyocytes were visualized by GFP, co-expressed from miRNA adenoviruses. Scale bar=20 μm.*, P<0.05. b. miR-1 attenuated PE-induced cardiomyocyte hypertrophy. After 48 hours of PE stimulation, miR-1-expressing NRVM were significantly smaller than controls. Images were captured and quantitatively analyzed by a blinded observer. Results were reproducible in three independent experiments.

FIG. 5. miRNA expression in dissociated cells. a, Increased fibrosis in two month old MHCα-CN hearts. was investigated using Masson's Trichrome Staining of histological sections, where staining indicates fibrotic tissue. Fibrotic area was calculated by quantitative measurement of fibrotic area in the histological sections. 3 hearts were analyzed per group. For each heart, percent fibrotic area was measured by a blinded observer in at least five adjacent sections. b, Cells were dissociated by collagenase perfusion and cardiomyocytes were collected by differential centrifugation. The cardiomyocyte fraction (CM) was greater than 90% pure as judged by microscopic examination. Non-cardiomyocytes were further fractionated into two populations by plating for 2 hours on tissue culture dishes. Adherent non-myocytes, consisting mainly of fibroblasts and endothelial cells, were labeled NM-A (non-myocytes, adherent). Non-adherent non-myocytes, which by microscopic examination contained primarily red blood cells, were labeled NM-B. miRNA expression was measured by qRTPCR and normalized to U6.*, P<0.05 compared with NTg control.

FIG. 6. Developmental pattern of miRNA expression. Expression of miR-1, -30b/c, -133a/b, and -208 was measured by qRTPCR at several developmental stages. These miRNAs were significantly upregulated during development. In heart failure, miRNA expression became more similar to the fetal pattern. E, embryonic days post-coitum. P, post-natal days. 2M, 2 months old.

### DETAILED DESCRIPTION OF THE INVENTION

The present invention relates to small-interfering nucleic acids and methods that are useful in the evaluation and therapy of heart failure. These compositions comprise small-interfering nucleic acids that may be used to inhibit expression of their target genes. An example of one small-interfering nucleic acid is an miRNA as herein described. Such small-interfering nucleic acid molecules are useful, for example, in providing compositions to prevent, inhibit, or reduce target gene expression in, for example, myocardium (e.g., myocardial tissue, myocardial cells). Thus, the present invention relates to using microRNAs (miRNAs) in methods for evaluation and therapy of heart disease and/or heart failure.

As described herein, Applicants measured the expression of 261 miRNAs in heart failure resulting from transgenic overexpression of calcineurin, a well accepted murine model of cardiac hypertrophy associated heart disease. In this investigation, 59 miRNAs were confidently detected in the heart and 11 miRNAs belonging to 6 families (miR-1, -15, -30, -133, -195, -208) were downregulated compared to non-transgenic control (Welch's t-test nominal p<0.05, false discovery rate <0.001). The results were validated by qRTPCR. There were no upregulated miRNAs identified in this investigation. Four of these miRNAs (miR-1, -30, -133, -208) were enriched in a purified cardiomyocyte preparation, compared to non-myocytes. Downregulation of these four miRNAs was reproduced in purified failing versus non-failing cardiomyocytes. This excluded artifactual downregulation from reduced myocyte fraction in failing hearts. The remaining two miRNAs (miR-15, and -195) were exclusively expressed in non-cardiomyocytes and did not changed in failing cardiomyocytes. Applicants,. used Affymetrix expression profiling to show that the predicted targets of these downregulated miRNAs were disproportionately upregulated compared to the entire transcriptome (Fisher's exact p<0.001). This indicates an association between downregulation of these miRNAs and upregulation of predicted target genes in heart failure. In particular, one target gene of the predominant cardiac microRNA miR-1 is calmodulin, a key regulator of calcium signaling. Applicants discovered that calmodulin and downstream calmodulin signaling to NFAT is regulated by miR-1 in cultured cardiomyocytes. Applicants' results indicate that altered expression of cardiomyocyte-enriched miRNAs contributes to abnormal gene expression in heart failure. Furthermore, the regulation of calmodulin and calcium signaling by miR-1 indicates a mechanism by which miR-1 regulates heart function.

As described herein, microRNA expression is altered in human heart disease. Applicants measured expression of 428 miRNAs in 67 human left ventricular samples belonging to control (n=10), ischemic cardiomyopathy (ICM, n=19), dilated cardiomyopathy (DCM, n=25), or aortic stenosis (AS, n=13) diagnostic groups. miRNA expression between disease and control groups was compared by ANOVA with Dunnett's post hoc test. Multiple testing was controlled for by estimating the false discovery rate. Out of 428 miRNAs measured, 87 were confidently detected. Forty-three were differentially expressed in at least one disease group. In supervised clustering, miRNA expression profiles correctly grouped samples by their clinical diagnosis, indicating that miRNA expression profiles are distinct between diagnostic groups. This was further supported by class prediction approaches, in which the class (control, ICM, DCM, AS) predicted by an miRNA-based classifier matched the clinical diagnosis 69% of the time (p<0.001). Applicants' data show that expression of many miRNAs is altered in heart disease, and that different types of heart disease are associated with distinct changes in miRNA expression. Applicants' discovery indicates the contribution of miRNAs to heart disease pathogenesis.

### Clinical Evaluation of Heart Disease

The present invention relates to methods useful for the clinical evaluation of heart disease based on the levels or occurrence of microRNA expression in myocardial cells. In some embodiments the invention relates to categorizing (classifying) a myocardial sample based on the occurrence or level microRNA expression in the sample. The methods involve assessing the sample for the occurrence or level of microRNA expression for at least one microRNA and categorizing using standard methods. In particular the methods involve categorizing a sample (for example, a myocardial tissue sample, or cells isolated therefrom) for the evaluation of disease (for example, heart disease) in a human. In some embodiments, evaluation involves assessing the risk of, or aiding in assessing the risk of, an individual having heart disease. In some embodiments evaluation involves diagnosing, or aiding in diagnosing, heart disease in an individual in need thereof.

Sample categorization (e.g., classifying a sample) can be performed for many reasons. For example, it may be desirable to classify a sample from an individual for any number of purposes, such as to determine whether the individual has a disease of a particular class or type so that the individual can obtain appropriate treatment. Other reasons for classifying a sample include predicting treatment response (e.g., response to a particular drug or therapy regimen) and predicting phenotype (e.g., the likelihood of heart disease). Thus, the applications of the invention are numerous and are not limited to the specific examples described herein. The invention can be used in a variety of applications to classify samples based on the patterns of microRNA expression of one or more genes in the sample.

For example, heart disease is a disease for which several classes or types exist (e.g., Ischemic Cardiomyopathy (ICM), Dilated Cardiomyopathy (DCM), Aortic Stenosis (AS)) and, many require unique treatment strategies. Thus, heart disease is not a single disease, but rather a family of disorders arising from distinct cell types (e.g., myocardial cells) by distinct pathogenetic mechanisms. The challenge of heart disease treatment has been to target specific therapies to particular heart disease types, to maximize effectiveness and to minimize toxicity. Improvements in heart disease categorization (classification) have thus been central to advances in heart disease treatment.

In one embodiment, the present invention was used to classify samples from individuals having heart disease as being either ICM, DCM, or AS samples. The present invention has been shown, as described herein, to accurately and reproducibly distinguish ICM, DCM, and AS samples, and to correctly classify test samples, for example via cross validation, as belonging to one or the other of these classes.

The present invention relates to classification based on the simultaneous expression monitoring of a large number of microRNAs using bead-based expression analysis technology. In some embodiments microRNA arrays or other methods developed to assess a large number of genes are used. Such technologies have the attractive property of allowing one to monitor multiple expression events in parallel using a single technique.

A further aspect of the invention includes assigning a biological sample (e.g., a myocardium sample) to a known or putative class (i.e., class prediction), for example a heart disease class such as ischemic cardiomyopathy, dilated cardiomyopathy, or aortic stenosis. by evaluating the occurrence or level of a microRNA in a sample, or microRNA expression patterns in the sample. Another embodiment of the invention relates to a method of discovering or ascertaining two or more classes from samples by clustering the samples based on microRNA expression values, to obtain putative classes (i.e., class discovery) or to reveal predicted classes. These embodiments are described in further detail below. In preferred embodiments, one or more steps of the methods are performed using a suitable processing means, e.g., a computer.

As used herein heart disease relates to the following non-limiting examples: Heart failure (congestive); Cardiomyopathies, such as Ischemic cardiomyopathy, Dilated cardiomyopathy, Hypertrophic cardiomyopathy, Restrictive cardiomyopathy, Alcoholic cardiomyopathy, Viral cardiomyopathy, Tachycardia-mediated cardiomyopathy, Stress-induced (takotsubo) cardiomyopathy, Amyloid cardiomyopathy, Arrhythmogenic right ventricular dysplasia, or unclassified cardiomyopathies, for example Left ventricular noncompaction or Endocardial fibroelastosis; or valvular heart disease, such as Aortic stenosis, Aortic regurgitation, Mitral stenosis, Mitral regurgitation, Mitral prolapse, Pulmonary stenosis, Pulmonary regurgitation, Tricuspid stenosis, or Tricuspid regurgitation.

In particular embodiments, class prediction is carried out using samples from individuals known to have the heart disease type or class being studied, as well as samples from control individuals not having the heart disease or having a different type or class of the heart disease. This provides the ability to assess microRNA expression patterns across the full range of disease phenotypes. Using the methods described herein, a classification model (e.g., linear discriminant function and support vector machine) is built with the microRNA expression levels from these samples. In one embodiment, this model is created from a set of two or more microRNAs whose expression pattern is associated with a particular disease class distinction (e.g., ICM, DCM, or AS) to be predicted.

A test sample assessed can be any sample (e.g., a myocardial tissue sample, also referred to as a myocardium sample, or cells isolated therefrom) that contains expressed microRNAs. A myocardial tissue sample can be obtained using an one of a variety of methods. For example, endomyocardial tissue biopsies can be obtained using methods known in the art (Grezeskowiak et al. 2003, Kittleson et al. 2004, Lowes et al. 2006, Moniotte et al. 2001).

Using the methods described herein, expression of numerous microRNAs can be measured simultaneously. The assessment of numerous genes can sometimes provide for a more accurate evaluation of a sample because there are more microRNA that can assist in classifying the sample. The microRNA expression levels are obtained, e.g., by using a bead-based system or a suitable array-based system (e.g., miRMAX microarray), and determining the extent of hybridization of the microRNA in the sample to the beads or the probes on the microarray. Once the microRNA expression levels of the sample are obtained, the levels are compared or evaluated against the model and the sample is classified. The evaluation of the sample determines whether the sample should be assigned to the particular heart disease class being studied or not.

In one embodiment, samples are classified into various types or classes of heart disease, in particular, ICM, DCM, or AS classes, based on the expression of certain microRNAs. MicroRNAs that are useful for determining the heart disease class of a test sample are also important in understanding pathogenesis of the heart disease class. In certain embodiments, one or more of these microRNAs provides therapeutic target(s) for treatment for the heart disease class. Hence, the present invention embodies methods for determining the relevant microRNAs for classification of samples as well as methods for determining the importance of a microRNA involved in the heart disease class as to which samples are being classified. In one embodiment, miR-1 is identified as important in classifying heart disease and is indicated to have a causal role in heart disease progression by regulating the expression of calmodulin activity. Consequently, the methods of the present invention also pertain to determining therapeutic target(s) based on microRNAs that are involved with the disease being studied.

In one embodiment the occurrence or level of microRNA in cells of an individual is greater than the level or occurrence of the microRNA in cells of a control individual. In another embodiment the occurrence or level of microRNA in cells of an individual is less than the level or occurrence of the microRNA in cells of a control individual. As used herein, the amount of the greater than and the amount of the less than is of a sufficient magnitude to, for example, facilitate distinguishing an individual from a control individual using the disclosed classification methods.

As used herein, expression pattern refers to the combination of occurrences or levels in a set of microRNAs of a sample. In assessing the similarity of two expression patterns, for example, a test expression pattern and a reference expression pattern, a comparison is made between the occurrence or level of the same microRNA (microRNA pair(s)) in the test and reference expression patterns for each microRNA pair.

In one embodiment the classification scheme involves building or constructing a model also referred to as a classifier or predictor, that can be used to classify samples to be tested (test samples) based on miRNA levels or occurrences. The model is built using reference samples (control samples) for which the classification has already been ascertained, referred to herein as a reference dataset comprising reference expression patterns. Hence, reference expression patterns are levels or occurrences of a set of one or more miRNAs in a reference sample (e.g., a reference myocardial tissue sample).

Once the model (classifier) is built, then a test expression pattern obtained from a test sample is evaluated against the model (e.g., classified as a function of relative miRNA expression of the sample with respect to that of the model). In some embodiments, evaluation involves identifying the reference expression pattern that most closely resembles the expression pattern of the test sample and associating the known disease class or type of the reference expression pattern with the test expression pattern, thereby classify (categorizing) the type of disease (e.g., heart disease) associated with the test expression pattern.

In some embodiments a portion (subset) of miRNAs can be chosen to build the model. In this example, not all available or detectable miRNAs are used to classify a test sample. The number of relevant miRNAs to be used for building the model can be determined by one of skill in the art. In one embodiment, a greedy search method (backward selection) with Support Vector Machine is used to determine a subset of miRNAs that can be chosen to build a model (e.g., Naïve Bayes and Logisitic regression) for heart disease class prediction.

A class prediction strength can also be measured to determine the degree of confidence with which the model classifies a sample to be tested. The prediction strength conveys the degree of confidence of the classification of the sample and evaluates when a sample cannot be classified. There may be instances in which a sample is tested, but does not belong to a particular class. This is done by utilizing a threshold wherein a sample which scores below the determined threshold is not a sample that can be classified (e.g., a “no call”). For example, if a model is built to determine whether a sample belongs to one of three heart disease classes, but the sample is taken from an individual who does not have heart disease, then the sample will be a “no call” and will not be able to be classified. The prediction strength threshold can be determined by the skilled artisan based on known factors, including, but not limited to the value of a false positive classification versus a “no call.”

Once a model is built, the validity of the model can be tested using methods known in the art. One way to test the validity of the model is by cross-validation of the dataset. To perform cross-validation, one of the samples is eliminated and the model is built, as described above, without the eliminated sample, forming a “cross-validation model.” The eliminated sample is then classified according to the model, as described herein. This process is done with all the samples of the initial dataset and an error rate is determined. The accuracy the model is then assessed. This model classifies samples to be tested with high accuracy for classes that are known, or classes have been previously ascertained or established through class discovery as discussed herein. Another way to validate the model is to apply the model to an independent data set, such as a new unknown test myocardial tissue sample. Other standard biological or medical research techniques, known or developed in the future, can be used to validate class discovery or class prediction.

An aspect of the invention also includes ascertaining or discovering classes that were not previously known, or validating previously hypothesized classes. This process is referred to herein as class discovery. This embodiment of the invention involves determining the class or classes not previously known, and then validating the class determination (e.g., verifying that the class determination is accurate). To ascertain classes that were not previously known or recognized, or to validate classes which have been proposed on the basis of other findings, the samples are grouped or clustered (for example, using unsupervised clustering) based on microRNA expression levels. The microRNA expression levels of a sample (e.g., a myocardial sample) from a microRNA expression pattern and the samples having similar microRNA expression patterns are grouped or clustered together. The group or cluster of samples identifies a class. This clustering methodology can be applied to identify any classes in which the classes differ based on microRNA expression.

Determining classes, such as heart disease classes, that were not previously known is performed by the present methods using a clustering routine. The present invention can utilize several clustering routines to ascertain previously unknown classes, such as Bayesian clustering, k-means clustering, hierarchical clustering, and Self Organizing Map (SOM) clustering (see, for example, U.S. Provisional Application No. 60/124,453, entitled, “Methods and Apparatus for Analyzing Gene Expression Data,” by Tayamo, et al., filed Mar. 15, 1999, and U.S. patent application Ser. No. 09/525,142, entitled, “Methods and Apparatus for Analyzing Gene Expression Data,” by Tayamo, et al., filed Mar. 14, 2000, the teachings of which are incorporated herein by reference in their entirety). Once the samples are grouped into classes using a clustering routine, the putative classes are validated. The steps for classifying samples (e.g., class prediction) can be used to verify the classes. As described herein, class discovery methods (unsupervised clustering) have been applied to a murine model of heart disease. Unsupervised clustering using microRNA expression profiles separated MHCα-CN and NTg mice into distinct classes (groups), indicating a systematic alteration of microRNA expression in this murine heart failure model. MicroRNA profiling of 2 month old MHCα-CN and non-transgenic (“NTg”) control hearts showed significantly altered expression (p<0.05) of eleven microRNAs belonging to seven families.

Classification of the sample gives a healthcare provider information about a classification to which the sample belongs, based on the analysis or evaluation of multiple genes. The methods can provide a more accurate assessment that traditional tests because multiple microRNAs are analyzed. The information provided by the present invention, alone or in conjunction with other test results, aids the healthcare provider in diagnosing the individual.

Also, the present invention provides methods for determining a treatment plan. Once the health care provider knows to which disease class the sample, and therefore, the individual belongs, the health care provider can determine an adequate treatment plan for the individual. For example, different heart disease classes often require differing treatments. As described herein, individuals having a particular type or class of heart disease can benefit from a different course of treatment, than an individual having a different type or class of heart disease. Properly diagnosing and understanding the class of heart disease of an individual allows for a better, more successful treatment and prognosis.

Other applications of the invention include ascertaining classes for or classifying persons who are likely to have successful treatment with a particular drug or therapeutic regiment. Those interested in determining the efficacy of a drug can utilize the methods of the present invention. During a study of the drug or treatment being tested, individuals who have a disease may respond well to the drug or treatment, and others may not. Often, disparity in treatment efficacy may be the result of genetic variations among the individuals. Samples are obtained from individuals who have been subjected to the drug being tested and who have a predetermined response to the treatment. A model can be built from a portion of the relevant microRNAs from these samples, for example, to provide a reference expression pattern. A sample to be tested can then be evaluated against the model and classified on the basis of whether treatment would be successful or unsuccessful. A company testing the drug could provide more accurate information regarding the class of individuals for which the drug is most useful. This information also aids a healthcare provider in determining the best treatment plan for the individual.

In some embodiments ascertaining classes for or classifying persons who are likely to have successful treatment with a particular drug or therapeutic regiment can be implemented for the following non-limiting drug classes, drugs, and therapeutic options. ACE inhibitors, such as Captopril, Enalapril, Lisinopril, or Quinapril; Angiotensin II receptor blockers, such as Valsartan; Beta-blockers, such as Carvedilol, Metoprolol, and bisoprolol; Vasodilators (via NO), such as Hydralazine, Isosorbide dinitrate, and Isosorbide mononitrate; Cardiac Glycosides, such as Digoxin; Antiarrhythmic agents, such as Calcium channel blockers, for example, Verapamil and Diltiazem or Class III antiarrhythmic agents, for example, Amiodarone, Sotalol or, defetilide; Diuretics, such as Loop diuretics, for example, Furosemide, Bumetanide, or Torsemide, Thiazide diuretics, for example, hydrochlorothiazide, Aldosterone antagonists, for example, Spironolactone or eplerenone; Statins, such as Simvastatin, Atrovastatin, Fluvastatin, Lovastatin, Rosuvastatin or pravastatin; Anticoagulation drugs, such as Aspirin, Warfarin, or Heparin; or Inotropic agents, such as Dobutamine, Dopamine, Milrinone, Amrinone, Nitroprusside, Nitroglycerin, or nesiritide. Other treatments are also applicable, such as Pacemakers, Defibrillators, Mechanical circulatory support, such as Counterpulsation devices (intraaortic balloon pump or noninvasive counterpulsation), Cardiopulmonary assist devices, or Left ventricular assist devices; Surgery, such as Cardiac transplantation, Heart-lung transplantation, or Heart-kidney transplantation; or Immunosuppressive agents, such as Myocophnolate mofetil, Sirolimus, Tacrolimus, Corticosteroids, azathiorine, Cyclosporine, Antithymocyte globulin, for example, Thymoglobulin or ATGAM, OKT3, IL-2 receptor antibodies, for example, Basilliximab or Daclizumab.

Another application of the present invention is classification of a sample from an individual to determine whether he or she is more likely to contract a particular disease or condition (for assessing the risk, or aiding in assessing the risk, of heart disease). For example, persons who are more likely to contract heart disease or high blood pressure can have genetic differences from those who are less likely to suffer from these diseases. A model, using the methods described herein, can be built from individuals who have heart disease or high blood pressure, and those who do not. Once the model is built, a sample from an individual can be tested and evaluated with respect to the model to determine to which class the sample belongs. An individual who belongs to the class of individuals who have the disease, can take preventive measures (e.g., exercise, aspirin, etc.).

In some embodiments after the samples are classified, the output (e.g., output assembly) is provided (e.g., to a printer, display or to another software package such as graphic software for display). The output assembly can be a graphical representation. The graphical representation can be color coordinated with shades of contiguous colors (e.g., blue, red, etc.). One can then analyze or evaluate the significance of the sample classification. The evaluation depends on the purpose for the classification or the experimental design. For example, if one were determining whether the sample belongs to a particular disease class, then a diagnosis or a course of treatment can be determined.

### Treatment of Heart Disease

The present invention also relates to methods useful for the treatment of heart disease based on the supplementation or inhibition of microRNA associated with heart disease. In some embodiments the supplementation or inhibition of microRNAs involves contacting a myocardial cell with a small-interfering nucleic acid that is identical to, or complementary to a microRNA associated with heart disease. In some embodiments the supplementation or inhibition of microRNAs involves contacting a myocardial cell with a small-interfering nucleic acid that is substantially similar to, or substantially complementary to a microRNA associated with heart disease.

### Small Interfering Nucleic Acids

The invention features small nucleic acid molecules, referred to as short interfering nucleic acid (siNA) that include, for example: microRNA (miRNA), short interfering RNA (siRNA), double-stranded RNA (dsRNA), and short hairpin RNA (shRNA) molecules. An siNA of the invention can be unmodified or chemically-modified. An siNA of the instant invention can be chemically synthesized, expressed from a vector or enzymatically synthesized as discussed herein. The instant invention also features various chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., molecule comprises one or more chemical modifications.

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In yet another embodiment, each strand of the siNA comprises about 16 to about 25 nucleotides. The target genes comprise, for example, sequences referred to in Table 1. These targets were predicted by sequence conservation in 4-5 vertebrate species (TargetScanS, Lewis et al, Cell 120:15-20, and www.targetscan.org). Applicants validated the targets listed in Table 5 by fusing the putative target sequences to a luciferase reporter, and confirming that specific miR expression reduced luciferase activity compared to expression of a negative control miR sequence.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

In another embodiment, a small interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the a-fetoprotein promoter.

One embodiment herein contemplates the use of gene therapy to deliver one or more expression vectors, for example viral-based gene therapy, encoding one or more small interfering nucleic acids, capable of inhibiting expression of genes associated with Heart Disease, for example Calmodulin. As used herein, gene therapy is a therapy focused on treating diseases, such as heart disease, by the delivery of one or more expression vectors encoding therapeutic gene products, including polypeptides or RNA molecules, to diseased cells. Methods for construction and delivery of expression vectors will be known to one of ordinary skill in the art.

Supplementation of miRNA Expression

The siNAs of the present invention, for example miRNAs, regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into an miRNA duplex, and further into a “mature” single stranded miRNA molecule. This mature miRNA guides a multiprotein complex, miRISC, which identifies target 3′ UTR regions of target mRNAs based upon their complementarity to the mature miRNA. In some embodiments the methods of the invention provide exogenous siNA to supplement the function of an miRNA downregulated in disease. In some embodiments downregulation of miRNA is causally related to the disease. For example, in some embodiments an siNA is delivered to cells to supplement the expression of an miRNA that is reduced in heart disease to treat the heart disease, wherein the siNA comprises a sequence substantially similar to the sequence of an miRNA. As used herein the sequence of an siNA is substantially similar to the sequence of an miRNA when the two sequences are identical, or sufficiently similar that the siNA is complementary, or sufficiently complementary, to a (at least one) target mRNA of the miRNA and is capable of hybridizing with and inhibiting the target mRNA. In some embodiments, an siNA sequence that is substantially similar to the sequence of an miRNA, is an siNA sequence that is identical to the miRNA sequence at all but 1, 2, 3, 4, 5, 6, 7, 8, 9, 10, 11, 12, 13, 14, 15, 16, 17, or 18 bases. In some embodiments, an siNA sequence that is substantially similar to the sequence of an miRNA, is an siNA sequence that is different than the miRNA sequence at all but up to one base. Any one of the siNAs (e.g., siRNA, miRNA, or shRNA) disclosed herein can be used for supplementing miRNA expression (activity). In some embodiments, an miRNA is supplemented by delivering an siRNA having a sequence that comprises the sequence, or a substantially similar sequence, of the miRNA. In still other embodiments, miRNA are supplemented by delivering miRNAs encoded by shRNA vectors. Such technologies for delivery exogenous microRNAs to cells are well known in the art. For example, the shRNA-based vectors encoding nef/U3 miRNAs produced in HIV-1-infected cells have been used to inhibit both Nef function and HIV-1 virulence through the RNAi pathway (Omoto S et al. Retrovirology. Dec. 15, 2004;1:44).

Inhibition of miRNA Function

An siNA (e.g., miRNA) inhibits the function of the mRNAs it targets and, as a result, inhibits expression of the polypeptides encoded by the mRNAs. Thus, blocking (partially or totally) the activity of the siNA (e.g., silencing the siNA) can effectively induce, or restore, expression of a polypeptide whose expression is inhibited (derepress the polypeptide). In one embodiment, derepression of polypeptides encoded by mRNA targets of an siNA is accomplished by inhibiting the siNA activity in cells through any one of a variety of methods. For example, blocking the activity of an miRNA can be accomplished by hybridization with an siNA that is complementary, or substantially complementary to, the miRNA, thereby blocking interaction of the miRNA with its target mRNA. As used herein, an siNA that is substantially complementary to an miRNA is an siNA that is capable of hybridizing with an miRNA, thereby blocking the miRNA's activity. In some embodiments, an siNA that is substantially complementary to an miRNA is an siNA that is complementary with the miRNA at all but 1, 2, 3, 4, 5, 6, 7, 8, 9, 10, 11, 12, 13, 14, 15, 16, 17, or 18 bases. In some embodiments, an siNA sequence that is substantially complementary to an miRNA, is an siNA sequence that is complementary with the miRNA at, at least, one base. Antisense oligonucleotides, including chemically modified antisense oligonucleotides—such as 2′ O-methyl, locked nucleic acid (LNA)—inhibit miRNA activity by hybridization with guide strands of mature miRNAs, thereby blocking their interactions with target mRNAs (Naguibneva, I. et al. Nat. Cell Biol. 8, 278-284 (2006), Hutvagner G et al. PLoS Biol. 2, e98 (2004), Orom, U. A., et al. Gene 372, 137-141 (2006), Davis, S. Nucleic Acid Res. 34, 2294-2304 (2006)). ‘Antagomirs’ are phosphorothioate modified oligonucleotides that can specifically block miRNA in vivo (Kurtzfeldt, J. et al. Nature 438, 685-689 (2005)). MicroRNA inhibitors, termed miRNA sponges, can be expressed in cells from transgenes (Ebert, M. S. Nature Methods, Epub Aug. 12, 2007). These miRNA sponges specifically inhibit miRNAs through a complementary heptameric seed sequence and an entire family of miRNAs can be silenced using a single sponge sequence. Other methods for silencing miRNA function (derepression of miRNA targets) in cells will be apparent to one of ordinary skill in the art.

### Treatment

One aspect of the invention contemplates the treatment of a individual having or at risk of having heart disease. As used herein an individual, also referred to as a subject, is a mammalian species, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects (individuals). The human subject may be a pediatric, adult or a geriatric subject.

As used herein treatment, or treating, includes amelioration, cure or maintenance (i.e., the prevention of relapse) of a disease (disorder). Treatment after a disorder has started aims to reduce, ameliorate or altogether eliminate the disorder, and/or its associated symptoms, to prevent it from becoming worse, or to prevent the disorder from re-occurring once it has been initially eliminated (i.e., to prevent a relapse).

The invention in other embodiments provides a pharmaceutical pack or kit comprising one or more containers filled with one or more of the ingredients of the pharmaceutical compositions of the invention. Associated with such container(s) can be various written materials (written information) such as instructions (indicia) for use, or a notice in the form prescribed by a governmental agency regulating the manufacture, use or sale of pharmaceuticals or biological products, which notice reflects approval by the agency of manufacture, use or sale for human administration.

The pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations. A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18th Ed. (1990).

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

An effective amount, also referred to as a therapeutically effective amount, of an siNA (for example, an siNA molecule capable of inhibiting or supplementing expression of miRNA associated with heart disease) is an amount sufficient to ameliorate at least one adverse effect associated with expression, or reduced expression, of the microRNA in a cell (for example, a myocardial cell) or in an individual in need of such inhibition or supplementation (for example, an individual having heart disease). The therapeutically effective amount of the siNA molecule (active agent) to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount.

Use of the small interfering nucleic acid-based molecules of the invention can lead to better treatment of the disease progression by affording, for example, the possibility of combination therapies (e.g., multiple small interfering nucleic acid molecules targeted to different microRNA, small interfering nucleic acid molecules coupled with known drugs (e.g., BetaBlockers), or intermittent treatment with combinations of small interfering nucleic acids and/or other chemical or biological molecules). The treatment of individuals with nucleic acid molecules can also include combinations of different types of nucleic acid molecules. In some embodiments therapeutic siNAs delivered exogenously are optimally stable within cells until translation of the target mRNA has been inhibited long enough to reduce the levels of the protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

The administration of the herein described small interfering nucleic acid molecules to a patient can be intravenous, intraarterial, intraperitoneal, intramuscular, subcutaneous, intrapleural, intrathecal, by perfusion through a regional catheter, or by direct intralesional injection. When administering these small interfering nucleic acid molecules by injection, the administration may be by continuous infusion, or by single or multiple boluses. The dosage of the administered nucleic acid molecule will vary depending upon such factors as the patient's age, weight, sex, general medical condition, and previous medical history. Typically, it is desirable to provide the recipient with a dosage of the molecule which is in the range of from about 1 pg/kg to 10 mg/kg (amount of agent/body weight of patient), although a lower or higher dosage may also be administered.

In some embodiments, it may be desirable to target delivery of a therapeutic to the heart, while limiting delivery of the therapeutic to other organs. This may be accomplished by any one of a number of methods known in the art. In one embodiment delivery to the heart of a pharmaceutical formulation described herein comprises coronary artery infusion. In certain embodiments coronary artery infusion involves inserting a catheter through the femoral artery and passing the catheter through the aorta to the beginning of the coronary artery. In yet another embodiment, targeted delivery of a therapeutic to the heart involves using antibody-protamine fusion proteins, such as those previously describe (Song E et al. Nature Biotechnology Vol. 23(6), 709-717, 2005), to deliver the small interfering nucleic acids disclosed herein.

While it is possible for the agents to be administered as the raw substances, it is preferable, in view of their potency, to present them as a pharmaceutical formulation. The formulations of the present invention for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof or deleterious to the inhibitory function of the active agent. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier, which constitutes one or more accessory ingredients. In general, the formulations are prepared by uniformly and intimately bringing into association the agent with the carrier(s) and then, if necessary, dividing the product into unit dosages thereof.

Formulations suitable for parenteral administration conveniently comprise sterile aqueous preparations of the agents, which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization. Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers.

Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant, and are conveniently presented in unit dose or multi-dose form, for example, in a sealed ampoule.

Having now generally described the invention, the same will be more readily understood through reference to the following Examples which are provided by way of illustration, and are not intended to be limiting of the present invention.

### Examples

### Example 1

### Downregulation of Cardiomyocyte-Enriched MicroRNAs Contributes to Altered Gene Expression in Heart Failure

MicroRNAs (MiRNAs) are novel regulators of mRNA abundance and translation, and altered miRNA expression has been implicated in oncogenesis and neural disease. (Ambros, V., Nature 431, 350-355 (2004); Di Leva, G., et al, Birth Defects Res C Embryo Today 78, 180-189 (2006); Bartel, D. P., Cell 116, 281-297 (2004); Meister, G., Nature 431, 343-349 (2004)). A number of miRNAs are highly enriched in the heart (Lagos-Quintana, M. et al., Curr Biol 12, 735-739 (2002); Baskerville, S., Rna 11, 241-247 (2005)), but the contribution of miRNAs to deranged gene expression in heart failure has not been previously examined. Here we describe downregulation of miR-1, -30b/c, -133a/b, and -208 in failing cardiomyocytes. Altered miRNA expression was associated with changes in the abundance and translation of the mRNAs of predicted target genes. We show that miR-1 negatively regulates calmodulin, a key regulator of cardiomyocyte growth and hypertrophy. In heart failure, miR-1 downregulation was associated with upregulation of calmodulin. Forced expression of miR-1 decreased calmodulin gene expression, downregulated calcium-calmodulin signaling through the calcineurin/NFAT pathway, and reduced cardiomyocyte hypertrophy in response to agonist. Our results suggest that altered miRNA expression contributes to abnormal gene expression in heart failure, and add to the growing evidence that miRNAs may be broadly involved in the pathogenesis of human disease.

Pathological changes in cardiomyocyte gene expression lead to impaired cardiomyocyte survival and contraction, ultimately resulting in heart failure (McKinsey, T. A., J Clin Invest 115, 538-546 (2005)). Given the broad effect of miRNAs on gene expression, we hypothesized that altered miRNA expression contributes to these changes in gene expression in the failing heart. To test this hypothesis, first we asked if miRNAs are differentially expressed in heart failure. As a model, we studied heart failure caused by transgenic, cardiac-restricted expression of constitutively activated calcineurin (MHCα-CN) (Molkentin, J. D. et al., Cell 93, 215-228 (1998)). Calcium signals are key regulators of cardiomyocyte growth and function, and calcineurin is an important transducer of these signals (Frey, N., McKinsey, T. A., Nat Med 6, 1221-1227 (2000)). Activation of calcineurin accompanies human heart failure, and calcineurin is required for cardiac hypertrophy (Wilkins, B. J., J Physiol 541, 1-8 (2002); Lim, H. W. et al., J Mol Cell Cardiol 32, 697-709. (2000)). Constitutive activation of calcineurin in MHCα-CN mice results in severe cardiac hypertrophy and failure (Molkentin, J. D. et al., Cell 93, 215-228 (1998)).

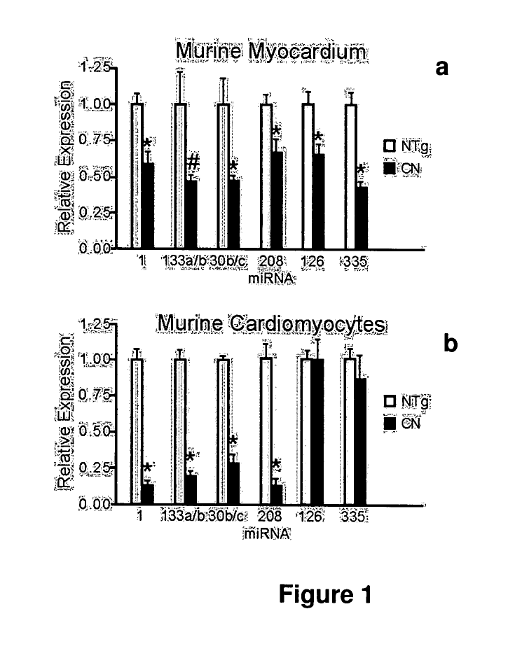

We used a previously validated bead-based method (Lu, J. et al., Nature 435, 834-838 (2005)) to profile the expression of 261 miRNAs in 2 month old MHCA-CN and non-transgenic (NTg) control hearts. 59 miRNAs had detectable expression (Table 2). Unsupervised clustering separated MHCα-CN and NTg mice into distinct groups, suggesting a systematic alteration of miRNA expression in this murine heart failure model. We found statistically significant (P<0.05, uncorrected Welch's P-value; and false discovery rate<0.001) downregulation of seven miRNAs belonging to six miRNA families (Table 1). There was no significantly upregulated miRNA. The cardiac-enriched miRNAs miR-1, miR-208, and miR-133b were downregulated. The other miR-133 family member, miR-133a, tended towards significant downregulation (P=0.051). Within the miR-30-5p family, all five members were either significantly downregulated (30b, 30e-5p, 30d; P<0.05) or tended towards significant downregulation (30c, 30a-5p; P<0.07). Measurement of mature miRNAs by quantitative RTPCR (qRTPCR) correlated closely with the bead-based profiling method (Table 1), and in each case confirmed significantly decreased expression (miR-1, miR-30b/c, miR-208, miR-126, and miR-335, P<0.05; FIG. 1a) or a tendency towards decreased expression (miR-133a/b; P=0.075; FIG. 1a). Rooij et al. recently described altered expression of a different set of microRNAs in the MHCα-CN heart failure model (van Rooij, E. et al., Proc Natl Acad Sci USA (2006)). Additional experiments will be needed to resolve these divergent results.

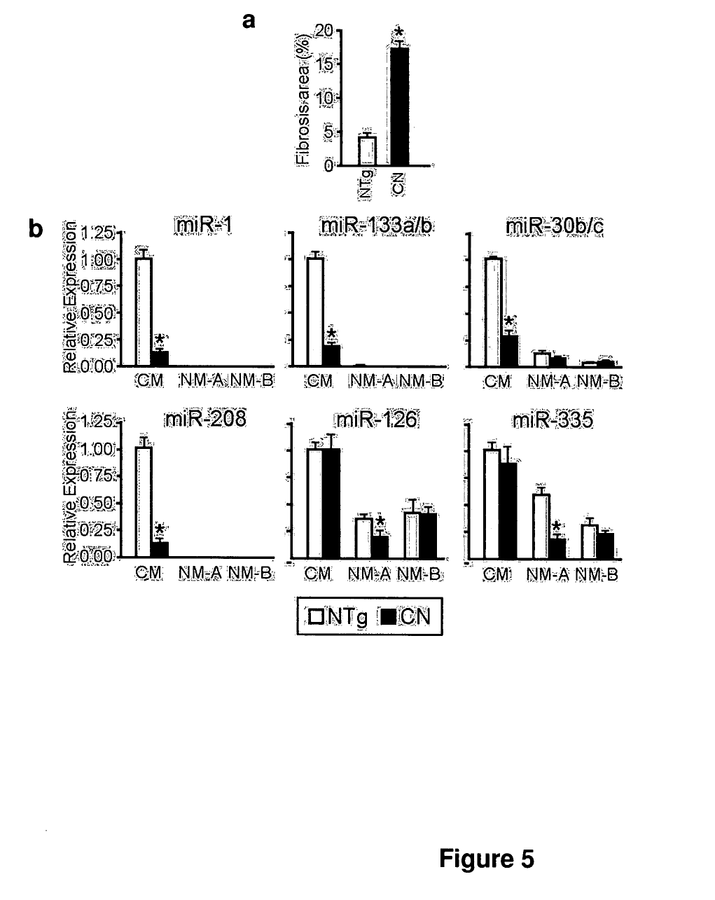

Heart failure is accompanied by significant myocardial fibrosis (FIG. 1a) and decreased proportion of cardiomyocytes to non-myocytes. In principle, decreased myocardial miRNA expression could be due to decreased expression in cardiomyocytes and/or to dilution of cardiomyocytes by non-myocytes. To distinguish these possibilities, we prepared enriched cardiomyocyte and non-myocyte populations (greater than 90% pure; FIG. 1a) by collagenase perfusion and differential centrifugation. Measurement of miRNA expression in these fractions by qRTPCR showed that the six miRNAs that were differentially expressed in heart failure by both bead-based assay and qRTPCR could be grouped into two classes: those that were substantially enriched in cardiomyocytes (miR-1, miR-133a/b, miR-30b/c, and miR-208) and those that were not (miR-126 and miR-335) (FIG. 5b). All four cardiomyocyte-enriched miRNAs showed significantly decreased expression in MHCα-CN compared to NTg cardiomyocytes (FIG. 1b; P<0.05). In contrast, the two miRNAs with less enrichment in cardiomyocytes were not changed between MHCα-CN and NTg cardiomyocytes (FIG. 1b), but were instead downregulated in non-cardiomyocytes (FIG. 1b).

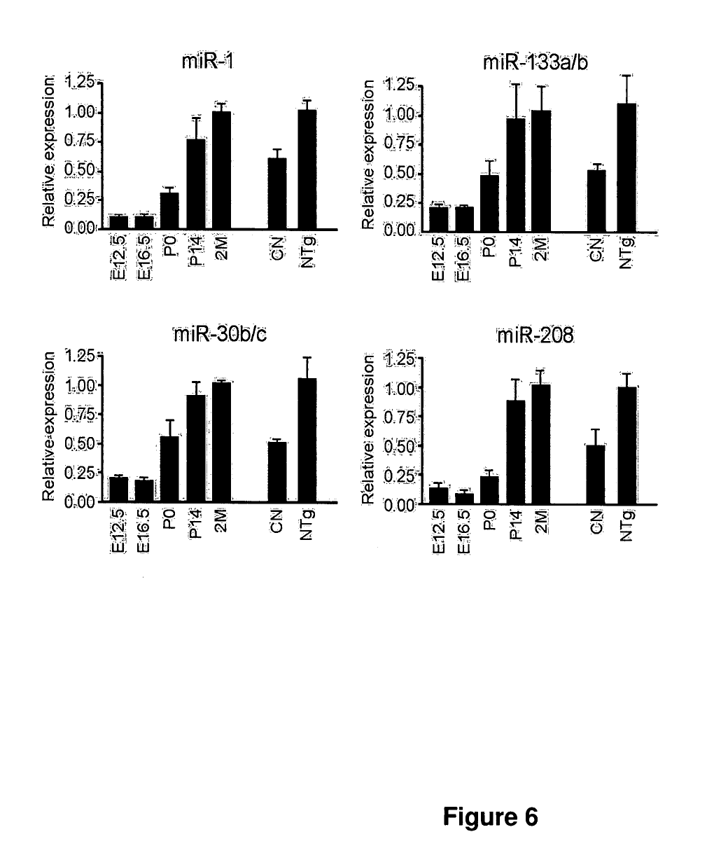

In failing cardiomyocytes, gene expression becomes more similar to the fetal expression profile (Izumo, S., et al., Proc Natl Acad Sci USA 85, 339-43. (1988); Komuro, I., et al., Circ Res 62, 1075-109. (1988)). To determine if this generalization also applies to miRNAs, we measured the level of cardiomyocyte-enriched miRNAs at several developmental time points (embryonic days (E) 12.5 and 16.5, and postnatal days (P) 0, 14, and two months). In each case, miRNA expression increased through fetal and perinatal development and into adulthood (FIG. 6) and decreased in heart failure. Thus, miRNA expression in the failing hearts indeed changed to become more similar to the fetal miRNA expression pattern.

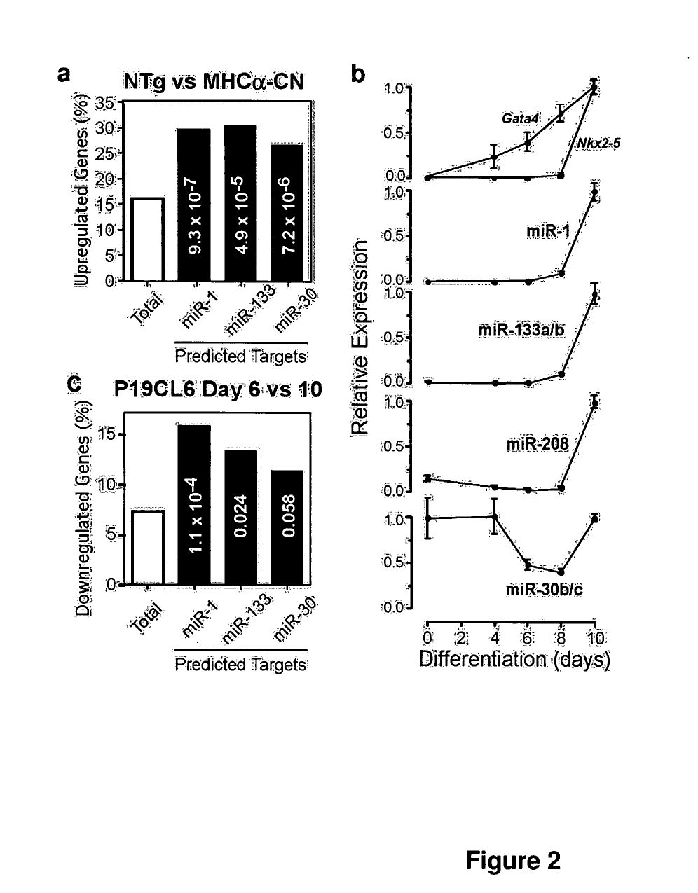

miRNAs influence gene expression by regulating mRNA abundance and/or mRNA translation (Meister, G., Nature 431, 343-349 (2004); Lim, L. P. et al., Nature 433, 769-773 (2005); Farh, K. K. et al., Science 310, 1817-1821 (2005)). Genome-wide transcriptional profiling has been used to detect the effect of miRNAs on mRNA transcript levels (Lim, L. P. et al., Nature 433, 769-773 (2005); Farh, K. K. et al., Science 310, 1817-1821 (2005)). If miRNAs regulate mRNA abundance in cardiomyocytes, then we hypothesized that downregulation of cardiac-enriched miRNAs would be associated with upregulation of predicted mRNA targets at a frequency greater than expected by random chance. To test this hypothesis, we used Affymetrix microarrays to obtain genome-wide measurements of mRNA levels in MHCα-CN and NTg hearts. Target genes of miR-1, miR-30, and miR-133 were predicted by conservation of their target regulatory sequences in the 3′ untranslated regions (UTRs) of 4-5 vertebrate species (TargetScanS algorithm (Lewis, B. P., et al., Cell 120, 15-20 (2005)). miR-208 target predictions were not available for this algorithm. In the whole transcriptome, out of 12,902 detectable genes, 2101 (16%) were upregulated at significance threshold of P<0.005 (uncorrected Welch's t-test). In comparison, out of 208 predicted miR-1 targets with detectable expression, 62 (30%) were upregulated. The likelihood that this or a larger proportion would occur in a random sampling of all detectable genes is 9.3×10⁻⁷ (Fisher's exact test; FIG. 2a). miR-30 and miR-133 targets were also upregulated at frequencies unlikely to occur by chance (FIG. 2a). These results were not sensitive to the specific significance threshold used to identify upregulated genes (Table 3).

The association between downregulation of miR-1, -30, and -133 and upregulation of their target genes suggests that altered expression of these miRNAs has broad effects on transcript abundance in the failing heart. To further support this interpretation, we asked if expression of these miRNAs is negatively related to target gene expression in an independent system. The multipotent embryonal carcinoma cell line P19CL6 differentiates into beating cardiomyocytes in the presence of DMSO (Habara-Ohkubo, A., Cell Struct Funct 21, 101 -110 (1996)). Cardiac differentiation follows a reproducible time course over 10 days that includes induction of the cardiac transcription factors Gata4 and Nkx2-5 (FIG. 2b). miR-1,-133, and -208 were highly upregulated between Day 6 and 10 of differentiation (FIG. 2b). Upregulation of miR-1 and -133 was associated with disproportionate downregulation of TargetScanS predicted target genes between Day 6 and 10 (FIG. 2c).

The effect of altered miR-1 and miR-133 expression on predicted targets could also be visualized qualitatively using gene expression density maps (Farh, K. K. et al., Science 310, 1817-1821 (2005)). Altered miRNA expression during P19CL6 cell differentiation is reflected in the pattern of expression of predicted mRNA target genes. Affymetrix microarrays were used to profile gene expression during P19CL6 differentiation. The expression profiles were used to generate the gene expression density maps). Briefly, at each time point a gene is assigned an expression rank, compared to the expression of the gene at other time points. A point is plotted using the gene's expression level (abscissa) and expression rank (ordinate). The density of points in the plot is color coded, with red representing the highest density, and blue the lowest. To control for random effects, the density map of randomly selected sets of genes (containing the same number as each miRNA target gene set) was subtracted. Gene expression density maps revealed increased miR-1 and miR-133a/b expression between Day 6 and Day 10 was associated with decreased expression rank of target genes (movement of red peak to lower expression rank).miR-30b/c expression was much less dynamic (2-fold change between Day 6 and 10; FIG. 2b). miR-30 predicted targets showed a trend towards disproportionate downregulation (P=0.058; FIG. 2c). Taken together, the negative relationship between miRNA level and target gene abundance in two independent systems suggests that these miRNAs broadly influence transcript abundance. These analyses do not address translational regulation, and thus the effect of altered miRNA level on gene expression in heart failure is likely to be even more pervasive.

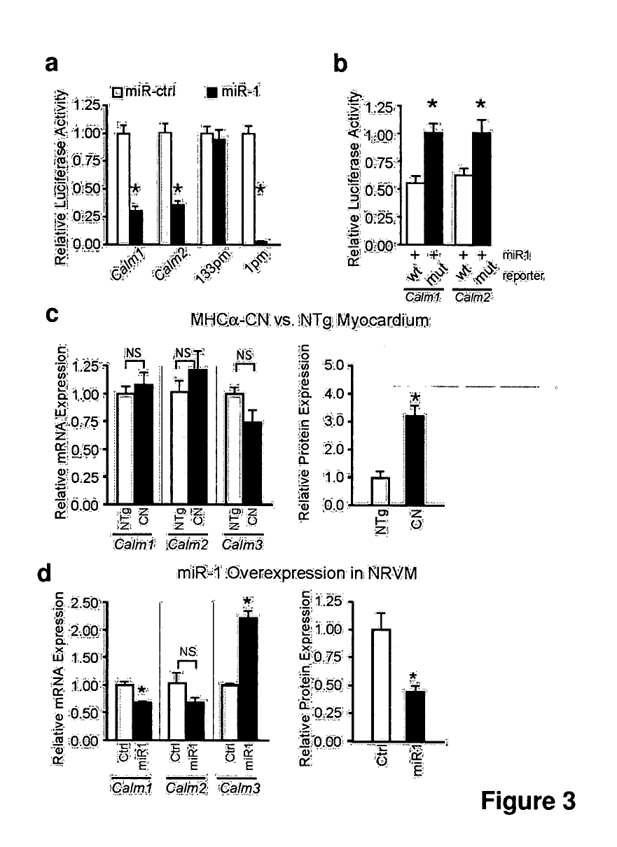

To investigate molecular mechanisms by which altered miRNA expression may influence the development of heart failure, we focused our attention on miR-1, the most highly expressed miRNA in the heart (Lagos-Quintana, M. et al., Curr Biol 12, 735-739 (2002)). Predicted targets of miR-1 include several that might contribute to heart failure pathogenesis, including genes encoding calmodulin. Calmodulin is a key regulator of calcium signaling, which has broad effects on cardiomyocyte growth, differentiation, and gene expression (Frey, N., McKinsey, T. A., Nat Med 6, 1221-1227 (2000)). Calmodulin is expressed from three non-allelic genes, Calm1, Calm2, and Calm3, which encode the identical protein. Calm1 and Calm2 account for 88% of calmodulin-encoding transcripts in the heart (based on signature sequencing tag counts (Jongeneel, C. V. et al., Genome Res 15, 1007-1014 (2005)). Intriguingly, each of these two genes contains a predicted miR-1 regulatory sequence (“seed match”) in its 3′ UTR that is conserved in 4 vertebrate species (FIG. 3a). Therefore, we hypothesized that miR-1 regulates Calm1 and Calm2. To test this hypothesis, we first asked if miR-1 would repress reporters in which the 3′ UTR of Calm1 or Calm2 was cloned downstream of luciferase. Compared with an unrelated control miRNA, miR-1 repressed the Calm1- and Calm2-containing reporters (FIG. 3a). The effect of miR-1 was blocked by mutation of the conserved miR-1 seed match sequences (FIG. 3b). These results validate Calm1 and Calm2 as miR-1 target genes.

To determine if miR-1 downregulation in heart failure was associated calmodulin upregulation, we measured calmodulin expression in MHCα-CN hearts. While Calm1 and Calm2 mRNA levels were not altered in MHCA-CN compared to NTg hearts, calmodulin protein was three-fold upregulated (P<0.05, FIG. 3c). Expression of Calm3 mRNA, which does not contain a miR-1 seed match sequence, was unchanged (FIG. 3c). Transgenic expression of calmodulin in a mouse model at this level was sufficient to cause severe cardiac hypertrophy and heart failure, (Gruver, C. L., et al., Endocrinology 133, 376-388 (1993); Obata, K. et al., Biochem Biophys Res Commun 338, 1299-1305 (2005)) suggesting that this degree of calmodulin upregulation is biologically important.

To further test the hypothesis that miR-1 negatively regulates calmodulin, we overexpressed miR-1 in neonatal rat ventricular cardiomyocytes (NRVMs). miR-1 overexpression did not affect Calm2 mRNA and reduced Calm1 mRNA by 32% (P<0.05; FIG. 3d). Calmodulin protein showed a greater reduction of 57% (P<0.05; FIG. 3d). This was not due to altered expression of the minor Calm3 transcript, which was upregulated (FIG. 3d). Decreased expression of calmodulin protein to a greater extent than mRNA suggests regulation at the level of translation. These data provide additional evidence that miR-1 negatively regulates calmodulin expression, independent of secondary effects related to heart failure.

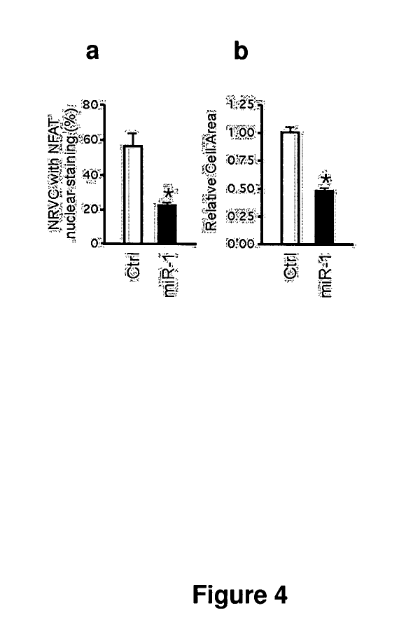

Calcium is a key regulator of cardiomyocyte growth and function, and many of the actions of calcium are mediated through its interaction with calmodulin (Frey, N., McKinsey, T. A., Nat Med 6, 1221-1227 (2000)). Free calmodulin is limiting in cardiomyocytes (Wu, X. et al., Cell Calcium (2006)), and therefore we hypothesized that miR-1 induced downregulation of calmodulin would attenuate calmodulin-dependent responses. Treatment of NRVMs with the α-adrenergic agonist phenylephrine (PE) increases calcium-calmodulin and thereby stimulates calcineurin, resulting in nuclear translocation of the transcription factor NFAT (Molkentin, J. D. et al., Cell 93, 215-228 (1998); Taigen, T., et al., Proc Natl Acad Sci USA 97, 1196-201. (2000)). Increased transcription of NFAT-dependent promoters is required for cardiac hypertrophy, and inhibition of this calcium-calmodulin/calcineurin/NFAT pathway blocks PE-stimulated NRVM hypertrophy (Taigen, T., et al., Proc Natl Acad Sci USA 97, 1196-201. (2000); Pu, W. T., Ma, Q. et al., Circ Res 92,725-731(2003)). Consistent with negative regulation of calmodulin by miR-1, miR-1 overexpression inhibited PE-induced NFAT nuclear translocation (FIG. 4b) and attenuated PE-induced cardiomyocyte hypertrophy (FIG. 4c). Collectively, these data suggest a model in which miR-1 negatively regulates the calcium-calmodulin/calcineurin/NFAT pathway and PE-induced hypertrophic responses by downregulating calmodulin.

This study shows that miRNA expression is altered in murine and human heart failure. Cardiomyocyte-enriched miRNAs miR-1, -30b/c, -133a/b, and -208 were highly downregulated in failing cardiomyocytes. Downregulation of these miRNAs was reflected in the transcriptome of failing hearts by disproportionate upregulation of predicted targets. Notable among targets of miR-l was calmodulin, which demonstrated an inverse relationship to miR-1 in the MHCα-CN heart failure model and which might provide a mechanistic link between altered miR-1 expression and the development of heart failure. Our data suggest that altered miRNA expression contributes to deranged gene expression in heart failure, and adds to the growing evidence that miRNAs may play a broad role in the pathogenesis of human disease.

### Methods

### Myocardial Samples

MHCα-CN transgenic mice were a kind gift from Jeffery Molkentin and previously described (Molkentin, J. D. et al., Cell 93, 215-228 (1998)). Human ischemic cardiomyopathy and dilated cardiomyopathy myocardial samples were from transplant recipients, and non-failing samples were from unused transplant donor hearts. These samples are described at www.cardiogenomics.org. Aortic stenosis samples were obtained at the time of aortic valve replacement. RNA was isolated from myocardial samples by homogenization in Trizol (Invitrogen). Protein was prepared from myocardial samples as previously described (Shioi, T. et al., Embo J 19, 2537-248. (2000)). Cardiomyocyte dissociation from adult hearts by collagenase perfusion was performed as described (Bodyak, N. et al., Nucleic Acids Res 30, 3788-3794 (2002)).

### Cell Culture

P19CL6 cells were cultured and induced to undergo cardiac differentiation as described previously (Habara-Ohkubo, A., Cell Struct Funct 21, 101-110 (1996); Ueyama, T., et al., Mol Cell Biol 23, 9222-9232 (2003)). NRVMs were prepared as described previously (Pu, W. T., Ma, Q. et al., Circ Res 92, 725-731 (2003)). NRVMs were stimulated with 20 μM phenylephrine.

### Gene Expression Analysis

miRNA expression profiles were obtained using a bead-based method as previously described (Lu, J. et al., Nature 435, 834-838 (2005)). 59 miRNAs were expressed above detection threshold in at least one sample (Table 2). Hierarchical clustering was performed with the complete linkage algorithm for both samples and features, using the 59 expressed miRNAs and the Pearson correlation coefficient as a similarity measure.

mRNA expression profiling was performed using the Affymetrix GeneChip 430 v2.0 as described (Bisping, E. et al., Proc Natl Acad Sci USA 103, 14471-14476 (2006)). miRNA target genes were predicted by TargetScanS version 2.1 for miR-1, miR-133, and miR-30. Gene expression and miRNA expression data will be submitted to the Gene Expression Omnibus (http://www.ncbi.nlm.nih.gov/geo/).

Quantitative reverse transcription PCR was performed on an ABI7300 Real-Time PCR System either sybr green or Taqman chemistry. Primer sequences or sources for qRTPCR assays are listed in Table 4. Gene expression was normalized to U6 or Gapdh for miRNAs and mRNAs, respectively. The miR-133a/b qRTPCR assay did not distinguish between miR-133a and miR-133b, and the miR-30b/c assay did not distinguish between miR-30b and -30c (data not shown). The miR-30b/c assay did not detect -30a, -30d, and -30e (data not shown).

Western blotting was performed using antibodies for Calmodulin (Upstate, 1:1,000) and Gapdh (Research Diagnostics, 1:5,000). NFATc3 immunostaining was performed using antibody from Santa Cruz (SC-8321, 1:200). Immunostained samples were imaged and analyzed by a blinded observer.

### Molecular Biology

Dual luciferase assays (Promega) were performed in transfected QBI293 cells (QBiogene; HEK293 subline). The luciferase vectors were generated from pMIR-REPORT (Ambion) by PCR subcloning of 3′ UTR fragments. miR-1 expression construct was generated by cloning the genomic fragment of miR-1 into pcDNA6.2-GW/emGFP-miR (Invitrogen). Negative control miRNA expression construct was pcDNA6.2-GW/emGFP-miR-neg (Invitrogen). This construct expresses a mature miRNA without known complementary sequence in vertebrate expressed sequences. Reporter assays represent the mean of four independent experiments, each in triplicate. Adenoviruses were generated using pAd/CMV/V5-DEST (Invitrogen) and purified on cesium chloride gradients. All primer sequences are in Table 4.

### Statistics

Unless otherwise indicated, two group comparisons were performed by non-parametric Wilcoxon rank sums test using JMP software v.5.1 (Cary, N.C,). For the bead-based miRNA assay, we used Welch's t-test to rank the significance of changes between groups. The false discovery rate (q-value) of each miRNA was calculated using the Significance Analysis of Microarrays (SAM) package (Storey, J. D. et al., Proc Natl Acad Sci USA 100, 9440-9445 (2003)). For Affyymetrix transcriptome data, we use the MAS 5 summary algorithm and linear scaling method to a median intensity chip. Probe sets below detection threshold across all the samples were excluded for further analysis. Remaining probe sets representing the same RefSeq transcript ID were averaged. Error bars indicate standard error of the mean.

TABLE 1
Differential miRNA expression in murine heart failure.

| | |
|---|---|
| Bead Array | qRTPCR |

| miRNA | Fold Change | p-val | Fold Change | p-val |
|---|---|---|---|---|
| miR-335 | −3.4 | 0.008 | −2.3 | 0.009 |
| miR-30b | −2.2 | 0.017 | −2.1^{A} | 0.009 |
| miR-1 | −1.9 | 0.018 | −1.6 | 0.028 |
| miR-30e-5p | −2.2 | 0.022 | | |
| miR-208 | −2.0 | 0.032 | −1.5 | 0.036 |
| miR-133b | −1.6 | 0.033 | −2.1^{B} | 0.075 |
| miR-30d | −1.7 | 0.048 | | |
| miR-16 | −1.6 | 0.051 | −1.3 | NS |
| miR-133a | −1.6 | 0.051 | −2.1^{B} | 0.075 |
| miR-126 | −1.9 | 0.052 | −1.5 | 0.028 |
| miR-15a | −1.9 | 0.052 | | |

miRNA expression was measured in MHCα-CN and NTg myocardium using a bead-based assay (Lu, J. et al., Nature 435, 834-838 (2005)). Mean expression was compared by Welch's t-test, and microRNAs were ranked by statistical score. In each case, false discovery rate was <0.0005. In selected cases, we independently measured gene expression in the same samples by qRTPCR.
^{A}qRTPCR assay measured both miR-30b and -30c, and did not detect miR-30a, -30d, or -30e.
^{B}qRTPCR assay measured both miR-133a and miR-133b.

TABLE 2
Bead-based profiling of miRNA expression in
2 month old Non-transgenic and MHCα-CN hearts.

| | | | | | | P-value | |
|---|---|---|---|---|---|---|---|
| | NTg | | MHCα-CN | | Fold- | (Welch's | |
| miRNA | avg | sem | avg | sem | change | t-test) | q-value |
| hmr-miR-335 | 352 | 55 | 105 | 23 | −3.4 | 0.008 | <0.001 |
| hmr-miR-30b | 1546 | 202 | 703 | 180 | −2.2 | 0.017 | <0.001 |
| hm-miR-1 | 5619 | 383 | 3030 | 644 | −1.9 | 0.018 | <0.001 |
| hmr-miR-30e-5p | 185 | 28 | 85 | 18 | −2.2 | 0.022 | <0.001 |
| hmr-miR-208 | 927 | 111 | 455 | 133 | −2.0 | 0.032 | <0.001 |
| hm-miR-133b | 1481 | 116 | 947 | 154 | −1.6 | 0.033 | <0.001 |
| hmr-miR-30d | 941 | 103 | 565 | 116 | −1.7 | 0.048 | <0.001 |
| hmr-miR-16 | 3020 | 324 | 1832 | 376 | −1.6 | 0.051 | <0.001 |
| hmr-miR-133a | 1358 | 112 | 844 | 172 | −1.6 | 0.051 | <0.001 |
| hmr-miR-126 | 1206 | 161 | 645 | 174 | −1.9 | 0.052 | <0.001 |
| hm-miR-15a | 300 | 47 | 162 | 36 | −1.9 | 0.052 | <0.001 |
| hmr-miR-125a | 178 | 14 | 104 | 26 | −1.7 | 0.058 | <0.001 |
| hmr-miR-30a-5p | 760 | 104 | 457 | 91 | −1.7 | 0.064 | <0.001 |
| hm-let-7g | 787 | 81 | 479 | 110 | −1.6 | 0.067 | <0.001 |
| hmr-miR-30c | 1154 | 124 | 747 | 143 | −1.5 | 0.073 | <0.001 |
| hmr-miR-26b | 940 | 115 | 519 | 159 | −1.8 | 0.077 | <0.001 |
| hmr-miR-21 | 80 | 12 | 310 | 88 | 3.9 | 0.078 | <0.001 |
| hmr-miR-130a | 335 | 43 | 195 | 51 | −1.7 | 0.081 | <0.001 |
| hmr-miR-30a-3p | 284 | 16 | 156 | 54 | −1.8 | 0.095 | <0.001 |
| hm-miR-199a* | 46 | 9 | 122 | 37 | 2.6 | 0.130 | 0.237 |
| hmr-miR-126* | 1134 | 87 | 676 | 225 | −1.7 | 0.132 | <0.001 |
| hmr-let-7d | 1124 | 75 | 744 | 189 | −1.5 | 0.137 | <0.001 |
| hmr-let-7f | 1126 | 111 | 737 | 192 | −1.5 | 0.141 | <0.001 |
| hmr-miR-99b | 119 | 14 | 165 | 22 | 1.4 | 0.141 | 0.385 |
| hmr-miR-199a | 48 | 6 | 117 | 37 | 2.4 | 0.156 | 0.244 |
| hmr-miR-24 | 355 | 42 | 537 | 97 | 1.5 | 0.159 | 0.230 |
| hmr-miR-214 | 77 | 6 | 186 | 65 | 2.4 | 0.192 | 0.237 |
| hm-miR-30e-3p | 343 | 24 | 231 | 69 | −1.5 | 0.201 | <0.001 |
| hmr-miR-29c | 561 | 88 | 367 | 106 | −1.5 | 0.206 | <0.001 |
| hmr-miR-106b | 147 | 15 | 106 | 25 | −1.4 | 0.216 | 0.127 |
| hmr-let-7a | 1872 | 209 | 1415 | 321 | −1.3 | 0.283 | <0.001 |
| hmr-miR-27b | 555 | 67 | 725 | 124 | 1.3 | 0.284 | 0.244 |
| hmr-miR-17-5p | 234 | 29 | 171 | 47 | −1.4 | 0.305 | 0.105 |
| hmr-miR-29b | 390 | 48 | 291 | 87 | −1.3 | 0.368 | 0.082 |
| hmr-miR-23b | 1057 | 112 | 1217 | 136 | 1.2 | 0.396 | 0.385 |
| hmr-miR-25 | 148 | 18 | 118 | 28 | −1.3 | 0.401 | 0.166 |
| h-miR-106a | 213 | 24 | 165 | 47 | −1.3 | 0.402 | 0.166 |
| hmr-miR-191 | 137 | 17 | 165 | 30 | 1.2 | 0.451 | 0.412 |
| hmr-miR-27a | 587 | 49 | 703 | 135 | 1.2 | 0.468 | 0.412 |
| hmr-let-7i | 161 | 20 | 131 | 34 | −1.2 | 0.481 | 0.166 |
| hmr-miR-26a | 1731 | 211 | 1494 | 280 | −1.2 | 0.524 | 0.166 |
| mr-miR-10b | 170 | 36 | 140 | 38 | −1.2 | 0.585 | 0.166 |
| hmr-miR-143 | 498 | 61 | 448 | 66 | −1.1 | 0.596 | 0.166 |
| hmr-miR-152 | 92 | 17 | 111 | 34 | 1.2 | 0.626 | 0.412 |
| hmr-miR-23a | 914 | 101 | 986 | 139 | 1.1 | 0.691 | 0.412 |
| hmr-miR-195 | 1602 | 180 | 1695 | 191 | 1.1 | 0.735 | 0.412 |
| hmr-miR-451 (j-mir-25) | 408 | 61 | 367 | 100 | −1.1 | 0.739 | 0.166 |
| hmr-miR-146a | 125 | 18 | 140 | 44 | 1.1 | 0.766 | 0.412 |
| m-miR-106a | 251 | 33 | 236 | 36 | −1.1 | 0.773 | 0.166 |
| hmr-miR-144 | 232 | 47 | 214 | 40 | −1.1 | 0.782 | 0.166 |
| hmr-miR-125b | 529 | 50 | 484 | 143 | −1.1 | 0.782 | 0.166 |
| hmr-miR-100 | 260 | 27 | 245 | 59 | −1.1 | 0.830 | 0.166 |
| hmr-miR-22 | 684 | 92 | 651 | 116 | −1.1 | 0.832 | 0.166 |
| hmr-miR-20a | 305 | 62 | 288 | 51 | −1.1 | 0.833 | 0.166 |
| hmr-miR-424 | 195 | 30 | 207 | 52 | 1.1 | 0.843 | 0.412 |
| hmr-miR-99a | 362 | 39 | 349 | 87 | −1.0 | 0.900 | 0.166 |
| hmr-let-7c | 1262 | 136 | 1222 | 327 | −1.0 | 0.917 | 0.166 |
| hmr-let-7b | 396 | 40 | 386 | 107 | −1.0 | 0.936 | 0.166 |
| hmr-miR-29a | 681 | 90 | 675 | 158 | −1.0 | 0.975 | 0.166 |

59 miRNAs were expressed above detection threshold in at least one sample.
q-value, the estimated false discovery rate.
sem, standard error of the mean.
n = 5 for NTg and 4 for MHCα-CN.

TABLE 3
miRNAs broadly influence gene expression in MHCα-CN myocardium

| | All Genes | | | miR-1 Target Genes | | | |
|---|---|---|---|---|---|---|---|
| Threshold | | | % | | | | Fisher's |
| P-value | up | total | up | up | total | % up | P-value |
| 0.0005 | 897 | 12902 | 7% | 29 | 208 | 14% | 2.90E−04 |
| 0.001 | 1211 | 12902 | 9% | 38 | 208 | 18% | 5.81E−05 |
| 0.005 | 2101 | 12902 | 16% | 62 | 208 | 30% | 9.32E−07 |
| 0.01 | 2605 | 12902 | 20% | 71 | 208 | 34% | 2.02E−06 |
| 0.05 | 3872 | 12902 | 30% | 103 | 208 | 50% | 3.90E−09 |
| | miR-133 Target Genes | | | miR-30 Target Genes | | | |
| Threshold | | | Fisher's | | | | Fisher's |
| P-value | total | % up | P-value | up | total | % up | P-value |
| 0.0005 | 127 | 11% | 2.05E−02 | 46 | 340 | 14% | 7.16E−06 |
| 0.001 | 127 | 16% | 2.05E−02 | 58 | 340 | 17% | 7.16E−06 |
| 0.005 | 127 | 31% | 4.93E−05 | 91 | 340 | 27% | 7.03E−07 |
| 0.01 | 127 | 41% | 8.11E−08 | 109 | 340 | 32% | 1.60E−07 |
| 0.05 | 127 | 53% | 8.46E−08 | 150 | 340 | 44% | 2.72E−08 |

mRNA expression in MHCα-CN and NTg myocardium were measured using Affymetrix GeneChips, and mean expression values were compared using Welch's t-test. The upregulated fraction among predicted targets of miR-1, miR-133, or miR-30 (TargetScanS predictions) was compared to the overall upregulated fraction. We found that fraction of upregulated targets was greater among predicted targets of these miRNAs than the overall upregulated fraction. This change was statistically significant as evaluated by Fisher's exact test. This result was not sensitive to the specific P-value threshold used to define upregulated genes.

TABLE 4
Oligonucleotides Sequences

| | | |
|---|---|---|
| Name | Species | Sequence/Source |

qRTPCR

| | | | |
|---|---|---|---|
| miR-1 | mrh | Ambion 30008 | |
| miR-16 | mrh | Ambion 30062 | |
| miR-30b/c | mrh | Ambion 30143 | |
| miR-126 | mrh | Ambion 30023 | |
| miR-133 | mrh | Ambion 30032 | |
| miR-208 | mrh | Ambion 30101 | |
| miR-335 | mrh | Ambion 30160 | |
| U6 | mrh | Ambion 30303 | |
| Calm1 up | m | GGGTCAGAACCCAACAGAAG | SEQ ID NO: 1 |
| Calm1 down | m | GCGGATCTCTCTTCGCTAT | SEQ ID NO: 2 |
| Calm1 up | r | GGCTGAACTGCAGGATATGA | SEQ ID NO: 3 |
| Calm1 down | r | AATGCCTCACGGATTTCTTC | SEQ ID NO: 4 |
| Calm2 up | m | GCAGAACTGCAGGACATGAT | SEQ ID NO: 5 |
| Calm2 down | m | CAAACACACGGAATGCTTCT | SEQ ID NO: 6 |
| Calm2 up | r | CGAGTCGAGTGGTTGTCTGT | SEQ ID NO: 7 |
| Calm2 down | r | GGTTGTTATTGTCCCATCCC | SEQ ID NO: 8 |
| Calm3 up | m | TACCTGGTGCTAACATCCCA | SEQ ID NO: 9 |
| Calm3 down | m | AAGATCACCGGCACATTACA | SEQ ID NO: 10 |
| Calm3 up | r | GAGACGGCCAGGTCAATTAT | SEQ ID NO: 11 |
| Calm3 down | r | AGAGGAGAGCGCAAGAAGAG | SEQ ID NO: 12 |
| Rodent GAPDH | mr | ABI 4308313 | |

Cloning

| | | | |
|---|---|---|---|
| Calm1 3′UTR up | h | CCAAGGGAGCATCTTTGGACTC | SEQ ID NO: 13 |
| Calm1 3′UTR down | h | TGCTTCTACCACACACAGCGAAG | SEQ ID NO: 14 |
| Calm1-miR1-50wt | | CTAGGTTCAAAGAAATTACAGTTTACGTCCATTC | |
| top | h | C AAGTTGTAAATGCTAGTCTT | SEQ ID NO: 15 |
| Calm1-miR1-50mut | | AGCTAAGACTAGCATTTACAACTTGAACTGGAC | |
| bot | h | G TAAACTGTAATTTCTTTGAAC | SEQ ID NO: 16 |
| Calm2 3′UTR up | h | TGTGCTTCTCTCCCTCTTTTCTCAC | SEQ ID NO: 17 |
| Calm2 3′UTR down | h | TAACTCTGCGTGGACTATGGACAG | SEQ ID NO: 18 |
| Calm2-miR1-50wt | | CTAGTGCTTATGGCACAATTTGCCTCAAAATCCA | |
| top | h | TT CCAAGTTGTATATTTGTTTTCCAA | SEQ ID NO: 19 |
| Calm2-miR1-50mut | | AGCTTTGGAAAACAAATATACAACTTGAACTGG | |
| bot | h | AT TTTGAGGCAAATTGTGCCATAAGCA | SEQ ID NO: 20 |
| mir1pm top | mrh | CTAGTGAATTCTACATACTTCTTTACATTCCA | SEQ ID NO: 21 |
| mir1pm bot | mrh | AGCTTGGAATGTAAAGAAGTATGTAGAATTCA | SEQ ID NO: 22 |
| mir133pm top | mrh | CTAGTGAATTCACAGCTGGTTGAAGGGGACCAA | SEQ ID NO: 23 |
| mir133pm bot | mrh | AGCTTTGGTCCCCTTCAACCAGCTGTGAATTCA | SEQ ID NO: 24 |
| mmu-mir-1-2 | m | CCCTCGAGCACTGGATCCATTACTCTTC | SEQ ID NO: 25 |
| mmu-mir-1-2 | m | GGTCTAGATTGGAATGGGGCTGTTAGTA | SEQ ID NO: 26 |

Phylogenetic conservation of miR1 seed match sequences within
the 3′UTR of the calmodulin encoding genes Calm1 and Calm2.
Seed and seed match sequences are in boldface. (Sequences 5′to 3′
unless otherwise noted)

| | | | |
|---|---|---|---|
| Calm1 | Human | UCAAAGAAAUUACAGUUUACGUCCAUUCCAAGUUGUAAAUGC | SEQ ID NO: 27 |
| | Mouse | UC-AGGAAAUGAUAAUUUACGUCCAUUCCAAGUUGUAAAUGC | SEQ ID NO: 28 |
| | Rat | UC-AGGAAAUGAUAAUUUACGUCCAUUCCAAGUUGUAAAUGC | SEQ ID NO: 29 |
| | Dog | UC-AGGAAAUGAUAAUUUACGUCCAUUCCAAGUUGUAAAUGC | SEQ ID NO: 30 |
| Calm2 | Human | AUGGCACAAUUUGCCUCAAAAUCCAUUCCAAGUUGUAUAUUU | SEQ ID NO: 31 |
| | Mouse | AUGGCACAAUUUGCCUCAAA-UCCAUUCCAAGUUGUAUAUUU | SEQ ID NO: 32 |
| | Rat | AUGGCACAAUUUGCCUCAAA-UCCAUUCCAAGUUGUAUAUUU | SEQ ID NO: 33 |
| | Dog | AUGGCACAAUUUGCCUCAAA-UCCAUUCCAAGUUGUAUAUUU | SEQ ID NO: 34 |

| | | |
|---|---|---|
| miR-1 | 3′-AAUGUAUGAAGAAAUGUAAGGU-5′ | SEQ ID NO: 35 |

abbreviations: m, mouse; r, rat; h, human

### Example 12

### miRNA Subset Selection for Class Predication

A subset of miRNAs was searched to best predict the failing heart to non-failing heart. Feature selection was done using a wrapper method that uses a classifier to evaluate attribute sets, but it employs cross-validation to estimate the accuracy of the learning scheme for each set. Specifically, greedy search method (backward selection) with Support Vector Machine was used using a popular machine learning package Weka version 3.5.6. Twenty miRs out of 78 detected miRs were identified. With the following 20 miRs, overall accuracy from cross validation was over 85%: let-7a, miR-1, miR-10b (h), miR-15a, miR-17-5p, miR-19a, miR-19b, miR-20a, miR-21, miR-23b, miR-27a, miR-28, miR-30d, miR-030e-5p, miR-106a (h), miR-106b, miR-126, miR-195, miR-208, and miR-222. Using this subset of 20 miRNAs, we applied other classification methods such as Naive Bayes and Logistic Regression with 3-fold cross validation, respectively achieving 88.8889% and 92.5926% correct classification rates for the two methods.

### Example 3

### MHCAα-CN Heart miRNA Profile

MicroRNA expression in a murine heart failure model was profiled, using a previously validated bead-array profiling platform (Lu, J. et al. Nature 435, 834-8 (2005). Initial studies centered on transgenic mice in which the myosin heavy chain alpha promoter was used to drive expression of activated calcineurin (MHCα-CN). Activation of calcineurin accompanies human heart failure, and calcineurin is required for cardiac hypertrophy. By two months of age, MHCα-CN mice uniformly have substantial cardiac hypertrophy and severe ventricular dysfunction (Lim et al, J. Mol Cell Cardiol, 32: 697-709. 2000). Unsupervised clustering using microRNA expression profiles separated MHCα-CN and NTg mice into distinct groups, suggesting a systematic alteration of microRNA expression in this murine heart failure model. MicroRNA profiling of 2 month old MHCα-CN and non-transgenic (“NTg”) control hearts showed significantly altered expression (p<0.05) of eleven microRNAs belonging to seven families (Table 4).

There were no significantly upregulated miRNAs. Within the miR-133 family, both miR-133a and miR-133b were significantly downregulated. Similarly, within the mir-30-5p family, all five members were either significantly downregulated (30b, 30e-5p, 30d; p<0.05) or tended towards significant downregulation (30c, 30a-5p; p<0.07). In miR-15/16 family, miR-15a and miR-16 were significantly decreased (p<0.05), and miR-15b was not detected. Quantitative RTPCR (qRTPCR) correlated closely with the bead-based profiling method (Table 11), and confirmed significantly decreased expression for six of seven miRNAs tested (FIG. 1b; p<0.05).

### Example 4

### Altered miRNA Expression in Cardiomyocytes

Quantitative RTPCR (qRTPCR) was used to validate differential expression of a subset 30 of microRNAs. Seven microRNA families were differentially expressed by bead-array, and relative expression for each was measured by qRTPCR. qRTPCR supported differential expression for several of these microRNAs (miR1, miR-30, miR-126, miR-133, miR-185, miR-208, and miR-335). Myocardium is composed of several cell types, the proportions of which change in heart failure. To determine if differential microRNA expression was due to altered composition of myocardium or to altered expression within cardiomyocytes, qRTPCR was used to measure microRNA expression in purified cardiomyocytes. Collagenase perfusion and differential centrifugation were used to dissociate and purify cardiomyocytes. The final cardiomyocyte preparation contained greater than 90% cardiomyocytes. qRTPCR measurement of microRNA expression in purified MHCα-CN versus NTg cardiomyocytes showed that altered microRNA expression occurred within cardiomyocytes for the four microRNAs that were most highly enriched in cardiomyocytes: miR-1, miR-30b, miR-133, and miR-208. All four cardiomyocytes enriched miRNAs showed significantly decreased expression in cardiomyocytes of MHCα-CN compared with NTg hearts (p<0.05). In contrast, two of three miRNAs (miR-126, miR-335) without cardiomyocytes-enrichment did not change significantly within cardiomyocytes but decreased in non-cardiomyocyte population.

### Example 5

### Developmental Expression Profile of 4 miRNAs

In cardiac hypertrophy and failure, gene expression becomes more similar to a fetal cardiac gene expression profile. To determine if this generalization also applies to microRNAs, the developmental expression profile of the four cardiomyocyte-enriched miRNAs (miR1, miR-30b, miR-133, and miR-208) at several developmental timepoints (E12.5, E16.5, PO, P14, and 2 months). In each of these four cases, miRNA expression increased through fetal development and into adulthood and decreased in heart failure. MicroRNA expression in the failing, transgenic hearts did change to become more similar to the fetal microRNA expression pattern.

### Example 6

### Evidence for Broad Effects of Altered MicroRNA Expression on Gene Transcript Levels in Heart Failure

MicroRNAs regulate gene expression by impairing target gene mRNA stability and translation to protein. Transcriptional profiling was used to investigate whether changes in microRNA expression were inversely correlated with expression of computationally predicted mRNA target genes. RNA from two month old MHCα-CN and NTg mice was used to probe Affymetrix gene expression arrays. For each micro RNA with differential expression in MHCα-CN hearts, a set of putative target genes was identified using a computational algorithm, TargetScanS. This algorithm identifies genes in which a microRNA “seed sequence” is conserved within the 3′ untranslated region (UTR) of 5 vertebrate species. The “Seed Sequence”, defined in Lewis et al, Cell 120:15-20, is the sequence at the 5′ end of the miR which is thought to define the sequence specificity of the miR. Within each microRNA target gene set, we computed the proportion of genes that showed differential expression inversely related to the miRNA. We used Fishers exact test to calculate the likelihood that the proportion would be found in a random sampling of genes from the dataset (Table 7). miRNAs regulate gene expression by impairing target gene mRNA stability and/or translation to proteins (Lim, H. W. et al., J Mol Cell Cardiol 32, 697-709. (2000); Izumo, S., et al., Proc Natl Acad Sci USA 85, 339-43. (1988); Lewis, B. P., et al., Cell 120, 15-20 (2005); Gruver, C. L., et al., Endocrinology 133, 376-88 (1993); Yang, L. L. et al., Circulation 109,255-61 (2004); Zhao, Y., Samal, E. et al., Nature 436, 214-20 (2005); Chen, J. F. et al., Nat Genet 38, 228-33 (2006); Jongeneel, C. V. et al., Genome Res 15, 1007-1014 (2005); Meister, G. & Tuschl, et al., Nature 431, 343-349 (2004)). If altered miRNA expression is physiologically significant, then miRNA downregulation might be associated with upregulation of predicted mRNA targets at a frequency greater than expected by random chance. The TargetScanS algorithm was used to predict targets of miR-1, miR-30b, and miR-133 (Lewis, B. P., et al., Cell 120, 15-20 (2005)); miR-208 target predictions were not available. Gene expression in MHCα-CN and nontransgenic control hearts was measured using Affymetrix microarrays, then calculated the proportion of upregulated genes among miR-1, miR-30b, or miR-133 targets, compared with the whole transcriptome. In the whole transcriptome, 1,211 genes (9.4%) were upregulated at significance threshold of P<0.001 out of 12,902 totally detectable genes. In comparison, among miR-1 targets 38 genes (18.3%) were upregulated out of 208 total genes. Using Fisher's exact test, the likelihood that this proportion would occur in a random sampling of genes from the whole transcriptome is 6×10̂5. The proportion of predicted miR-30b and miR-133 targets that are upregulated was also highly significant. These data suggest that downregulation of these miRNAs has broad effects on transcript abundance in the failing heart. This method does not address translational regulation, and thus the effect of miRNAs on gene expression is likely to be even more pervasive.

### Example 7

### miR-1 Regulates Calmodulin Expression Level

Predicted miR-1 targets include several that could contribute to heart failure pathogenesis. Among these are Calm1 and Calm 2, the primary calmodulin isoforms in the heart, accounting for 88% of calmodulin-encoding transcripts (based on signature sequencing tag counts) (Jongeneel, C. V. et al., Genome Res 15, 1007-1014 (2005)). Calm1 and Calm2 were investigated as to whether they are biological miR-1 targets by cloning their 3′UTR into downstream of luciferase. The resulting constructs were significantly repressed by miR-1, compared with an unrelated control miRNA. A 50 bp region of the 3′ UTR encompassing the phylogenetically conserved miR-1 seed match sequence was sufficient to confirm sensitivity to miR-1, and mutation of this sequence abolished miR-1 sensitivity. miR-1 downregulation in MHCα-CN hearts was associated with significant, three-fold upregulation of calmodulin protein but not mRNA. Transgenic expression of calmodulin at this level was sufficient to cause severe cardiac hypertrophy, suggesting that this degree of calmodulin upregulation likely is biologically important (Gruver, C. L., et al., Endocrinology 133, 376-88 (1993)). Overexpression of miR-1 in cultured neonatal rat ventricular cardiomyocytes resulted in significant downregulation of calmodulin mRNA and protein. These data indicate that miR-1 can directly influence calmodulin expression in at least some cellular contexts.

Calcium-calmodulin signaling is a key regulator of cardiomyocyte hypertrophy and failure. Downstream targets include calcineurin, protein kinase C, and calcium-calmodulin kinase II. Thus, our data indicate that miR-1 controls expression of an important regulator of cardiac growth and function. Our data also indicate the possible existence of a calcineurin-calmodulin positive feedback loop mediated by miR-1, wherein calcineurin activation downregulates miR-1, which upreglates calmodulin, thereby increasing calcineurin activation.

### Example 8

### Target Gene Expression is Inversely Related to Cognate miRNA Expression

Additional predicted miR-1 targets may contribute to heart failure pathogenesis. Among these are the genes which encode connexin43 (Cx43), endothelin-1 (Ednl), and histone deacetylase 4 (Hdac4). We cloned the 3′ UTR of these genes downstream of luciferase and measured the effect of co-transfected miR-1 on luciferase activity was measured. MiR-1 significantly downregulated expression of luciferase in these constructs. Abundance of luciferase transcripts was unaltered, as determined by Northern blotting, suggesting that miR1 primarily regulates these genes at the translational level.

### Example 9

### Methods

### Myocardial Samples

MHCα-CN transgenic mice were a kind gift from Jeffery Molkentin and previously described (Lu, J. et al. Nature 435, 834-8 (2005)). Human ischemic cardiomyopathy and dilated cardiomyopathy myocardial samples were from transplant recipients, and non-failing samples were from unused transplant donor hearts. Myocardial samples were all obtained from the LV free wall. These samples are described at www.cardiogenomics.org. RNA was isolated from MiRNAs in Heart Failure myocardial samples by homogenization in Trizol (Invitrogen). Protein was prepared from myocardial samples as previously described (Shioi, T. et al. EMBO J 19, 2537-2548 (2000)). The failing and non-failing AS samples were obtained from myocardium excised at the time of aortic valve replacement.

Cardiomyocyte dissociation by collagenase perfusion was performed as described (Bodyak, N. et al., Nucleic Acids Res 30, 3788-3794 (2002)).

### Gene Expression Analysis

miRNA expression profiles was obtained using a bead-based method as previously described (Lim, H. W. et al., J Mol Cell Cardiol 32, 697-709. (2000)). We excluded miRNAs with signal intensity below threshold in all samples, as previously described (Lim, H. W. et al., J Mol Cell Cardiol 32, 697-709. (2000)). This filtering reduced the total number of miRNAs into 59, as shown in Table 9. Hierarchical clustering was performed with the complete linkage algorithm for both samples and features, using the 59 expressed miRNAs and the Pearson correlation as a similarity measure.

mRNA expression profiling was performed using the Affymetrix 430 v2.0 GeneChip as described (Bisping, E. et al., Proc Natl Acad Sci USA (2006)). miRNA target genes were predicted by TargetScanS for miR-1, miR-133, and miR-30b. This algorithm identifies genes in which an miRNA “seed sequence” is conserved within the 3′ untranslated region (UTR) of 4-5 vertebrate species (Zhao, Y., Samal, E. et al., Nature 436, 214-20 (2005)).

Quantitative Real Time PCR was performed using ABI7300 Real-Time PCR System using Power SYBR green master mix (Applied Biosystems). Primer sequences or sources for qRTPCR assays are listed in Table 10. For miRNAs, gene expression is relative to U6. For mRNAs, gene expression is relative to Gapdh. The qRTPCR assay for miR-133 did not distinguish miR-133a from miR-133b. Western blotting was performed using antibodies for Calmodulin (Upstate, 1:1,000 dilution) and Gapdh (Research Diagnostics, 1:5,000 dilution).

### Molecular Biology

Dual luciferase assays (Promega) were performed in transfected QBI293 cells (QBiogene; HEK293 subline). The luciferase vectors were generated from pMIRREPORT (Ambion) by PCR subcloning of 3′ UTR fragments. miR-1 expression construct was generated by cloning the genomic fragment of miR-1 into pcDNA6.2-GW/emGFP-miR (Invitrogen). Negative control miRNA expression construct is pcDNA6.2-GW/emGFP-miR-neg (Invitrogen) and expresses a mature miRNA without known complementary sequence in vertebrate expressed sequences. Adenoviruses were generated using pAd/CMV/V5-DEST (Invitrogen). All primer sequences in Table 10.

### Statistics

Two group comparisons were performed by Welch's t-test. Error bars indicate S.E.M.

TABLE 5
Validated MicroRNA Targets Relevant to Heart Failure

| | | Fold down- |
|---|---|---|
| microRNA | Target Gene | regulation by MiR |
| miR-1 | Endothelin-1 | 1.69 |
| miR-1 | Calmodulin-1 | 3.25 |
| miR-1 | Calmodulin-2 | 2.80 |
| miR-1 | Brain Derived | 1.69 |
| | Neurotrophic Factor | |
| miR-1 | Histone Deacetylase 4 | 1.91 |
| miR-1 | ETS-1 | 2.75 |
| miR-1 | Connexin 43 | 2.03 |
| miR-208 | Titin | 1.26 |
| miR-208 | Eya4 | 1.56 |

TABLE 6
MicroRNAs With Altered Expression in Heart Failure

| microRNA | Mean fold-change | p-value |
|---|---|---|
| miR-335 | −3.4 | 0.01 |
| miR-30b | −2.2 | 0.02 |
| miR-1 | −1.9 | 0.02 |
| miR-30e-5p | −2.2 | 0.02 |
| miR-208 | −2.0 | 0.03 |
| miR-133B | −1.6 | 0.03 |
| miR-30d | −1.7 | 0.05 |
| miR-16 | −1.6 | 0.05 |
| miR-133a | −1.6 | 0.05 |
| miR-126 | −1.9 | 0.05 |
| miR-15a | −1.9 | 0.05 |
| miR-125a | −1.7 | 0.06 |
| miR-30a-5p | −1.7 | 0.06 |
| let-7g | −1.6 | 0.07 |
| miR-30c | −1.5 | 0.07 |

TABLE 7
Broad Effects of Altered MicroRNA Expression
on Gene Transcript Levels in Heart Failure.

| | Target | Non-Target | |
|---|---|---|---|
| | Transcripts | Transcripts | Fisher's Exact |
| miR | (up/total) | (up/total) | Test P value |
| miR-1 | 38/208 | 1173/11521 | 0.000058 |
| miR-133 | 20/127 | 1191/11584 | 0.0205 |
| miR-30 | 58/340 | 1153/11409 | 0.0000072 |

Targets of miR-1, 30, and 133 were predicted by TargetScanS. Mir-208 is not included in the 5-species predictive algorithm because it is not known to be conserved in the 5 genomes used by TargetScanS.

TABLE 8
Sequence of Members of Cardiac-Enriched miR
Family Members

| | | |
|---|---|---|
| hsa-mir-1-2 | UGGAAUGUAAAGAAGUAUGUA | SEQ ID NO: 36 |
| hsa-mir-1-1 | UGGAAUGUAAAGAAGUAUGUA | SEQ ID NO: 37 |
| hsa-mir-133a- | UUGGUCCCCUUCAACCAGCUGU | SEQ ID NO: 38 |
| 1 | | |
| hsa-mir-133a- | UUGGUCCCCUUCAACCAGCUGU | SEQ ID NO: 39 |
| 2 | | |
| hsa-mir-133b | UUGGUCCCCUUCAACCAGCUA- | SEQ ID NO: 40 |
| hsa-miR-30d | UGUAAACAUCCCCGACUGGAAG-- | SEQ ID NO: 41 |
| hsa-miR-30e- | UGUAAACAUCCUUGACUGGA---- | SEQ ID NO: 42 |
| 5p | | |
| hsa-miR-30a- | UGUAAACAUCCUCGACUGGAAG-- | SEQ ID NO: 43 |
| 5p | | |
| hsa-miR-30a- | -CUUUCAGUCGGAUGUUUGCAGC- | SEQ ID NO: 44 |
| 3p | | |
| hsa-miR-30b | UGUAAACAUCCUACACUC--AGCU | SEQ ID NO: 45 |
| hsa-miR-30c | UGUAAACAUCCUACACUCUCAGC- | SEQ ID NO: 46 |
| hsa-miR-208 | AUAAGACGAGCAAAAAGCUUGU | SEQ ID NO: 47 |

TABLE 9
59 miRNA detected in the heart by bead-based method

| | Fold | |
|---|---|---|
| miRNA | Change | P-value |
| miR-335 | −3.4 | 0.008 |
| miR-30b | −2.2 | 0.017 |
| miR-1 | −1.9 | 0.018 |
| miR-30e-5p | −2.2 | 0.022 |
| miR-208 | −2.0 | 0.032 |
| miR-133b | −1.6 | 0.033 |
| miR-30d | −1.7 | 0.048 |
| miR-16 | −1.6 | 0.051 |
| miR-133a | −1.6 | 0.051 |
| miR-126 | −1.9 | 0.052 |
| miR-15a | −1.9 | 0.052 |
| miR-125a | −1.7 | 0.058 |
| miR-30a-5p | −1.7 | 0.064 |
| let-7g | −1.6 | 0.067 |
| miR-30c | −1.5 | 0.073 |
| miR-26b | −1.8 | 0.077 |
| miR-21 | 3.9 | 0.078 |
| miR-130a | −1.7 | 0.081 |
| miR-30a-3p | −1.8 | 0.095 |
| miR-199a* | 2.6 | 0.130 |
| miR-126* | −1.7 | 0.132 |
| let-7d | −1.5 | 0.137 |
| let-7f | −1.5 | 0.141 |
| miR-99b | 1.4 | 0.141 |
| miR-199a | 2.4 | 0.156 |
| miR-24 | 1.5 | 0.159 |
| miR-214 | 2.4 | 0.192 |
| miR-30e-3p | −1.5 | 0.201 |
| miR-29c | −1.5 | 0.206 |
| miR-106b | −1.4 | 0.216 |
| let-7a | −1.3 | 0.283 |
| miR-27b | 1.3 | 0.284 |
| miR-17-5p | −1.4 | 0.305 |
| miR-29b | −1.3 | 0.368 |
| miR-23b | 1.2 | 0.396 |
| miR-25 | −1.3 | 0.401 |
| h-miR-106a | −1.3 | 0.402 |
| miR-191 | 1.2 | 0.451 |
| miR-27a | 1.2 | 0.468 |
| let-7i | −1.2 | 0.481 |
| miR-26a | −1.2 | 0.524 |
| miR-10b | −1.2 | 0.585 |
| miR-143 | −1.1 | 0.596 |
| miR-152 | 1.2 | 0.626 |
| miR-23a | 1.1 | 0.691 |
| miR-195 | 1.1 | 0.735 |
| miR-451 (j-mir-25) | −1.1 | 0.739 |
| miR-146a | 1.1 | 0.766 |
| m-miR-106a | −1.1 | 0.773 |
| miR-144 | −1.1 | 0.782 |
| miR-125b | −1.1 | 0.782 |
| miR-100 | −1.1 | 0.830 |
| miR-22 | −1.1 | 0.832 |
| miR-20a | −1.1 | 0.833 |
| miR-424 | 1.1 | 0.843 |
| miR-99a | −1.0 | 0.900 |
| let-7c | −1.0 | 0.917 |
| let-7b | −1.0 | 0.936 |
| miR-29a | −1.0 | 0.975 |

TABLE 10
Sequence of oligonuleoides used in this study

| Name | Sequence/Source | |
|---|---|---|
| miR-1 | Ambion 30008 | |
| miR-16 | Ambion 30062 | |
| miR-30b | Ambion 30143 | |
| miR-126 | Ambion 30023 | |
| miR-133a | Ambion 30032 | |
| miR-208 | Ambion 30101 | |
| miR-335 | Ambion 30160 | |
| U6 | Ambion 30303 | |
| Mouse Calm1 | GGGTCAGAACCCAACAGAAG | SEQ ID NO: 1 |
| Forward | | |
| Mouse Calm1 | GCGGATCTCTTCTTCGCTAT | SEQ ID NO: 2 |
| Backward | | |
| Mouse Calm2 | GCAGAACTGCAGGACATGAT | SEQ ID NO: 5 |
| Forward | | |
| Mouse Calm2 | CAAACACACGGAATGCTTCT | SEQ ID NO: 6 |
| Backward | | |
| Rat Calm1 | GGCTGAACTGCAGGATATGA | SEQ ID NO: 3 |
| Forward | | |
| Rat Calm1 | AATGCCTCACGGATTTCTTC | SEQ ID NO: 4 |
| Backward | | |
| Rat Calm2 | CGAGTCGAGTGGTTGTCTGT | SEQ ID NO: 7 |
| Forward | | |
| Rat Calm2 | GGTTGTTATTGTCCCATCCC | SEQ ID NO: 8 |
| Backward | | |
| Rodent GAPDH | ABI 4308313 | |

TABLE 11

| | |
|---|---|
| Bead method | qRTPCR |

| miRNA | Fold Change | p-val | Fold Change | p-val |
|---|---|---|---|---|
| miR-335 | −3.4 | 0.01 | −2.3 | 0.001 |
| miR-30b | −2.2 | 0.02 | −2.1 | 0.04 |
| miR-1 | −1.9 | 0.02 | −1.6 | 0.009 |
| miR-30e-5p | −2.2 | 0.02 | | |
| miR-208 | −2.0 | 0.03 | −1.5 | 0.02 |
| miR-133b | −1.6 | 0.03 | −2.1^{A} | 0.05 |
| miR-30d | −1.7 | 0.05 | | |
| miR-16 | −1.6 | 0.05 | −1.3 | NS |
| miR-133a | −1.6 | 0.05 | −2.1^{A} | 0.05 |
| miR-126 | −1.9 | 0.05 | −1.5 | 0.02 |
| miR-15a | −1.9 | 0.05 | | |
| miR-125a | −1.7 | 0.06 | | |
| miR-30a-5p | −1.7 | 0.06 | | |
| let-7g | −1.6 | 0.07 | | |
| miR-30c | −1.5 | 0.07 | | |

^{A}qRTPCR does not distinguish miR-133 isoforms.

### Example 10

### Assessment of miRNA Expression Profiles in Four Diagnostic Groups: ICM, DCM, AS and Non-failing Controls

### Methods

### Patients

Human left ventricle samples belonged to four diagnostic groups (control, ICM, DCM, and AS). End-stage ICM and DCM samples were from explanted hearts of transplant recipients. ICM and DCM patients on mechanical assist devices or with ejection fraction (EF) greater than 45% were excluded. Control samples were from unused transplant donor hearts, with a maximal time between cardiectomy and sample collection of two hours. Aortic stenosis (AS) samples were obtained at the time of aortic valve replacement. Myocardial samples were snap frozen in liquid nitrogen. Areas of fibrosis visible on gross inspection were excluded from the collected myocardial samples. Samples were from Brigham and Women's Hospital (Boston, Mass.) and Georg August University (Gottingen, Germany), and collected under protocols approved by the respective Institutional Review Boards.

miRNA Measurement

RNA was isolated from myocardial samples by homogenization in Trizol (Invitrogen, Carlsbad, Calif.). miRNA profiling was performed using a high-throughput platform based on hybridization to optically addressed beads, as previously described (Lu J, Getz G, et al., Nature 435: 834-838, 2005). Quantitative reverse transcription PCR (qRTPCR) was performed on an ABI7300 Real-Time PCR System using Sybr Green chemistry and commercial primers (Applied Biosystems, Foster City, Calif.).

### Bioinformatics and Statistical Analysis

Expression threshold was set at average signal intensity detected in samples without input miRNA. miRNA expression data by bead-based assay was normalized by the locally weighted smooth spline (LOWESS) method on log-scaled raw data (Venables W N, Ripley B D. Modern applied statistics with S. 2002). After normalization, all expression values were transformed to linear scale for statistical comparisons. The miRNA expression heat map was constructed by unsupervised hierarchical clustering of miRNAs.

Oneway Analysis of Variance (ANOVA) with Dunnett's post hoc test was performed for signal intensity of each miRNA. We used Significance Analysis of Microarray software (Tusher V G, Tibshirani R, et al., Proc Natl Acad Sci USA 98: 5116-5121, 2001) to estimate the false discovery rate for each pairwise comparison between disease group and control. Supervised clustering by miRNA expression profiles was performed using Fisher's linear discriminant analysis (Venables W N, Ripley B D. Modern applied statistics with S. 2002). Class prediction was performed using a classifier derived by a supervised machine learning technique (support vector machine, SVM) implemented for the R statistical language in CRAN package e1071 (Cortes C, Vapnik V., Machine Learning 20: 273-297, 1995).

Statistical analysis was performed using JMP IN version 5 statistical software (SAS Institute, Cary, N.C.). Values are reported as mean±standard deviation.

### Results

### Patient Characteristics

We purified total RNA from left ventricular myocardium of 67 patients belonging to four diagnostic groups (control, n=10; ICM, n=19; DCM, n=25; and AS, n=13). Patient characteristics are summarized in Table 12. ICM and DCM patients had severely depressed EF and elevated pulmonary capillary wedge pressures. 10 out of 13 AS patients had preserved EF (EF>40%). ICM patients were more likely to be male than controls. AS patients were significantly older than controls. ICM, DCM, and AS patients were more likely to be treated with medications and to have comorbid conditions than controls.

Differential Expression of miRNAs in Human Heart Disease

Applicants profiled expression of 428 miRNAs using a high throughput bead-based platform (Lu J, Getz G, et al., Nature 435: 834-838, 2005). This platform was previously validated using Northern blotting (Lu J, Getz G, et al., Nature 435: 834-838, 2005). They further confirmed the reliability of this platform by measuring expression of nine miRNAs in 46 samples using qRTPCR. The nine miRNAs were selected to span the range of high, medium, and low intensity signals. There was strong correlation between the bead-based and qRTPCR measurements in eight out of nine miRNAs (Table 14). Within these 46 samples, seven miRNAs were differentially expressed in disease compared to control by bead-based measurements. This was supported by qRTPCR measurement in six of the seven cases.

Eighty-seven miRNAs were expressed above detection threshold in greater than 75% of samples (Table 13). An overview of these data is displayed in a heat map and a dendrogram, with samples grouped horizontally by diagnosis, and miRNAs arranged vertically by similarity of expression to one another. Applicants focused our attention on these confidently detected miRNAs so that the downstream analysis was based on the most reliable expression data.

To identify individual miRNAs with altered expression in heart disease, Applicants compared miRNA expression between each disease group and the control group, using ANOVA with Dunnett's post-hoc test (significance threshold P<0.05). To address multiple concurrent testing, we also required the estimated false discovery rate to be less than 5%. Out of 87 miRNAs that were confidently detected, 43 were differentially expressed in at least one disease group (Table 13), suggesting that expression of many miRNAs is altered in heart disease. Differential expression of these miRNAs persisted after multiple regression to control for sex and body mass index. Likewise, correction for age did not influence differential expression between ICM or DCM and control. AS patients were significantly older than controls, and the age distributions did not permit controlling for this confounding variable by multiple regression.

Among the miRNAs with known cardiac-enriched expression (miRNA-1, -133, and -208), miR-1 was downregulated in DCM and AS, and tended to be downregulated in ICM (P=0.054). Expression of miR-133 and miR-208 were not significantly changed. The most strongly upregulated miRNA was miR-214, which increased 2-2.8 fold in all three disease groups (Table 13). Upregulation of miR-214 may contribute to cardiac hypertrophy, as cardiomyocyte overexpression of miR-214 induced cardiomyocyte hypertrophy (van Rooij E, Sutherland L B, et al., Proc Natl Acad Sci USA 2006). The most strongly downregulated miRNA family was miR-19. The two miR-19 family members miR-19a and miR-19b were downregulated 2-2.7 fold in DCM and AS, but not in ICM (Table 13).

miRNA Expression Profiles are Distinct between Diagnostic Classes

The pattern of altered miRNA expression in each disease group was distinct. Differential expression of 13 miRNAs was specific to AS, while 8 miRNAs were differentially expressed in cardiomyopathy groups (ICM+DCM) and did not overlap with those altered in AS (Table 13). This suggests that altered expression of some miRNAs reflects distinct disease mechanisms or disease stage in AS compared to cardiomyopathy samples.

To further assess whether miRNA expression profiles were distinct between diagnostic groups, we performed supervised clustering of samples. Using Fisher's linear discriminant analysis (Venables W N, Ripley B D. Modern applied statistics with S. 2002), miRNA expression profiles segregated the samples by etiological diagnosis (ICM, DCM, or AS) with 100% accuracy. These results indicate that each form of heart disease is characterized by an miRNA expression profile that is sufficiently distinctive to allow construction of a discriminator that can accurately cluster samples by diagnostic group.

To further investigate the association of heart disease classes with distinct miRNA expression profiles, we asked if the expression profiles could predict clinical diagnosis. Applicants used a supervised learning technique, SVM, to develop an miRNA-based classifier. After training on the set of 67 samples, the SVM-derived classifier assigned class labels that matched the clinical diagnosis in all cases. Next, we performed cross-validation studies in which 45 randomly chosen samples were used for SVM training, and the resulting classifier was applied to the remaining 22 samples. This procedure was repeated 20,000 times. The classes assigned by the SVM-generated classifier matched the clinical diagnosis 69.2%±3.8% of the time. The likelihood of achieving this performance by chance was less than 0.001, estimated by SVM training on datasets in which the sample labels were randomly permuted (20,000 datasets with randomly permuted sample labels, each with 20,000 cross-validation studies). These results suggest that miRNA expression profiles are sufficiently distinct between disease classes to predict clinical diagnosis with moderate success. These data also provide proof-of-correct evidence that miRNA expression profiles would be useful as biomarkers for other class prediction problems, such as prediction of prognosis or treatment response.

In this work, Applicants report the first extensive genome-wide profiling of miRNA expression in human heart disease. They found that expression of many miRNAs changed significantly in diseased myocardium. Multiple independent lines of evidence corroborate our profiling data. First, miRNA expression measurements correlated closely between bead-based and qRTPCR platforms (Table 14). Second, the study yielded results largely concordant with previously reported findings. Olson and colleagues used northern blotting to compare miRNA expression in six DCM samples to four controls (van Rooij E, Sutherland L B, et al., Proc Natl Acad Sci USA 2006). They reported on 11 miRNAs, 10 miRNAs that were detectably expressed on our platform. The two studies were in agreement for 9 of the 10 miRNAs. Northern analysis suggested that miR-208 expression was not altered in human ICM (van Rooij E, Sutherland L B, et al., Science 2007), consistent with our data (Table 13). miR-1 was recently reported to be downregulated in four different murine models of cardiac hypertrophy or failure (Care A, Catalucci D, et al., Nat Med 13: 613-618, 2007; Sayed D, Hong C, et al., Circ Res 2007), consistent with Applicants' finding of miR-1 downregulation in AS and DCM.

However, not all studies are in agreement. While miR-133 was not significantly changed in our study, it was reported to be downregulated in hypertrophic cardiomyopathy and in dilated atrial myocardium (Care A, Catalucci D, et al., Nat Med 13: 613-618, 2007). They found that miR-1 was downregulated in ICM, while Yang and colleagues recently reported it was upregulated in ICM (Yang B. et al., Nat Med 13: 486-491, 2007). An oligonucleotide microarray study of a small number of samples (DCM, n=6; control, n=4) was recently published, and overall there was low concordance between data sets (Thum T, Galuppo P, et al., Circulation 116: 258-267, 2007). These divergent findings may reflect differences in tissues sampled (endocardial versus transmural; atrial versus ventricular), diagnostic groups studied, heterogeneity in human myocardial samples, systematic differences in the manner in which control or diseased samples are collected, and sample size differences that can lead to false discovery as well as false negatives (Tibshirani R., BMC Bioinformatics 7: 106, 2006). Additional miRNA profiling studies with larger sample numbers and careful attention to patient characteristics and details of tissue procurement will be necessary to resolve these differences.

miRNAs are emerging as important post-transcriptional regulators of gene expression, with each miRNA predicted to regulate hundreds of target genes (Ambros V., Nature 431: 350-355, 2004; Bartel D P., Cell 116: 281-297, 2004). A growing body of data indicates that miRNAs are key regulators of cardiac development, contraction, and conduction (Care A, Catalucci D, et al., Nat Med 13: 613-618, 2007; Sayed D, Hong C, et al., Circ Res 2007; van Rooij E, Sutherland L B, et al., Proc Natl Acad Sci USA 2006; van Rooij E, Sutherland L B, et al., Science 2007; Yang B, Lin H, et al., Nat Med 13: 486-491, 2007; Zhao Y, Ransom J F, et al., Cell 2007; Zhao Y, Samal E, et al., Nature 436: 214-220, 2005). In this study, we found that expression of many miRNAs was altered in human heart disease, albeit the magnitude of expression changes was generally small. These changes are not a simple epiphenomenon of end-stage heart disease, because AS patients had at the same time the most distinctive miRNA expression profile and largely compensated ventricular function. Rather, these miRNA changes likely contribute to heart disease pathogenesis by mediating pathological changes in gene expression. The distinctive pattern of miRNA expression changes between heart disease etiologies further suggests that miRNAs contribute to etiology-specific gene expression changes. The functional significance of these broad but often subtle changes in miRNA expression will need to be studied in model systems where levels of one or more miRNAs can be finely manipulated.

One long term goal of expression profiling studies is to develop expression signatures that can be used in clinically relevant classification problems, such as prognosis or prediction of drug responsiveness (Golub T R, et al., Science 286: 531-537, 1999; Kittleson M M, et al., Circulation 110: 3444-3451, 2004). In this study, we showed the miRNA expression profiles can classify samples by etiological diagnosis. This provides proof-of-concept that miRNA expression profiles may be useful in other more challenging and clinically relevant class prediction problems, and supports further studies of miRNAs as potential biomarkers for determining prognosis and response to therapy.

Analysis of human myocardial tissue is complicated by limited availability and by biological variability arising from differences in age, gender, body habitus, medications, co-morbidities, and individual course of disease. Intergroup differences in confounding variables was an important limitation of this study. We were able to control for some of these variables (gender, BMI, and age in DCM and ICM). However, we were unable to control for co-morbidities or medication use. In addition, AS patients were significantly older than cardiomyopathy patients or controls. We cannot exclude the possibility that the age difference contributed to altered miRNA expression in the AS group. However, we found no significant correlation between miRNA expression and age for any of the differentially expressed miRNAs within the control group, suggesting that miRNA expression does not systematically vary with age through adult life.

This study demonstrated that expression of many miRNAs is altered in human heart disease, and that the pattern of alteration differs by underlying disease etiology. This dataset of human miRNA expression in nonfailing and diseased hearts will guide further studies on the contribution of miRNAs to heart disease pathogenesis.

TABLE 12
Clinical Characteristics of the Study Subjects

| | Control | ICM | DCM | AS |
|---|---|---|---|---|
| Sample number | 10 | 19 | 25 | 13 |
| Age -- decades | 5.8 ± 1.4 | 6.6 ± 0.6 | 6.0 ± 1.5 | 8.6 ± 0.7 |
| Male sex -- no. (%) | 6 (60%) | 17 (89%) | 17 (68%) | 6 (46%) |
| BMI -- kg/m² | 24.2 ± 4.7 | 25.4 ± 5.1 | 23.5 ± 2.9 | 26.9 ± 3.0 |
| Medical History -- no (%) | | | | |
| Hypertension | 6 (60%) | 11 (58%) | 5 (20%) | 7 (50%) |
| DM | 1 (10%) | 11 (58%) | 5 (20%) | 3 (21%) |
| Atrial fibrilation | 0 (0%) | 3 (16%) | 9 (36%) | 3 (21%) |
| Cardiac function | | | | |
| LVEF - % | 65.0 ± 5.0† | 20.0 ± 7.5 | 15.9 ± 7.5 | 55.8 ± 16.9 |
| PCWP -- mmHg | N/A | 20.2 ± 8.6 | 20.5 ± 7.9 | 29.8 ± 4.3†† |
| Medication - no. (%) | | | | |
| ACE inhibitor/AR blockers | 0 (0%) | 14 (74%) | 20 (80%) | 8 (62%) |
| Beta-blockers | 2 (20%) | 10 (53%) | 15 (60%) | 7 (54%) |
| Diuretics | 0 (0%) | 17 (90%) | 19 (76%) | 10 (77%) |
| Digoxin | 0 (0%) | 11 (58%) | 15 (60%) | 3 (23%) |

†only available for three patients
††only available for seven patients
BMI, body mass index; DM, diabetes mellitus; LVEF, left ventricular ejection fraction; PCWP, pulmonary capillary wedge pressure. ACE, angiotensin converting enzyme; AR, angiotensin II receptor.

TABLE 13
Confidently detected miRNAs.

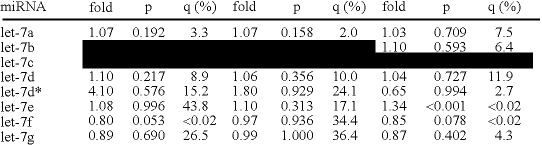

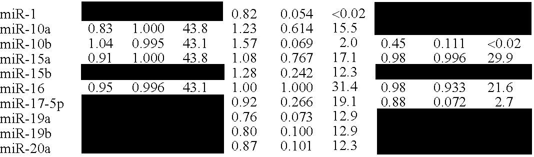

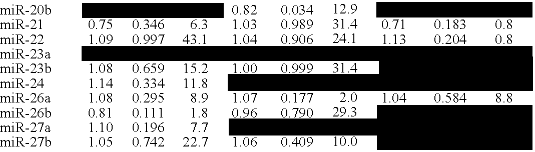

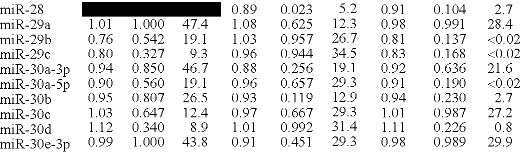

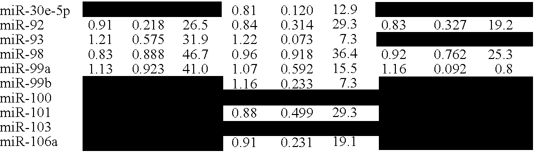

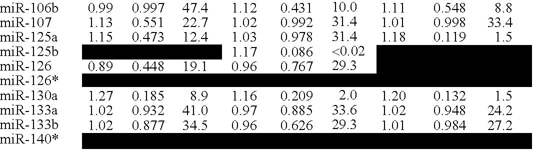

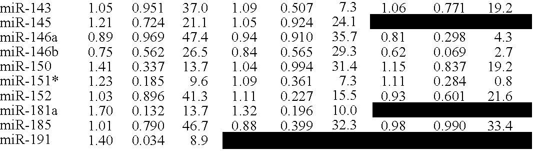

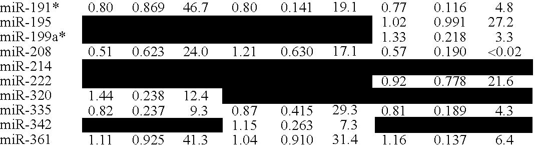

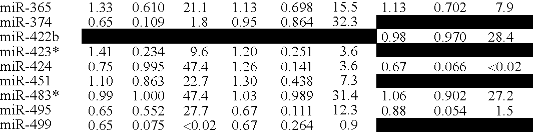

The miRNAs listed in this table were expressed above detection threshold in more than 75% of samples. Orange boxes indicate significant differences from control (P < 0.05, ANOVA with Dunnett's post-hoc testing; and false discovery rate (q) < 5%).

TABLE 14
Correlation between bead-based and qRTPCR platforms

| | Average | Pearson | |
|---|---|---|---|
| | expression in bead- | correlation | |
| miRNA | based assay | coefficient | p-value |
| miR-1 | 8654 ± 1820 | 0.497 | <0.001 |
| miR-30b† | 1800 ± 170 | −0.201 | 0.203 |
| miR-103 | 126 ± 21 | 0.458 | 0.003 |
| miR-126* | 685 ± 185 | 0.720 | <0.001 |
| miR-133a§ | 1210 ± 141 | 0.583 | <0.001 |
| miR-140* | 196 ± 48 | 0.575 | <0.001 |
| miR-191 | 98 ± 25 | 0.608 | <0.001 |
| miR-199a* | 85 ± 29 | 0.753 | <0.001 |
| miR-208 | 133 ± 89 | 0.909 | <0.001 |

Correlation between platforms in 46 samples representing the four diagnostic groups. miRNAs were chosed to include low, medium, and high expression values, displayed as mean ± sd. Relative miRNA expression values by qRTPCR were normalized to total input RNA.‡
†RTPCR assay measured both miR-30b and -30c. The assay did not detect miR-30a, -30d, or -30e. Expression levels of miR-30b and miR-30c were quite similar in the bead-based assay (r = 0.860, p < 0.001, Pearson correlation coefficient).
§qRTPCR did not distinguish miR-133a and miR-133b. Expression levels of miR-133a and miR-133b were quite similar in the bead-based assay (r = 0.898, p < 0.001, Pearson correlation coefficient).
‡U6 was not used as an internal control because its expression changed significantly in heart disease.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. Thus, for example, reference to “a cell” includes a plurality of such cells, reference to “the miRNA” is a reference to one or more miRNAs and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

1. (canceled)

2. A method for diagnosing, or aiding in diagnosing, heart disease in an individual in need thereof, comprising
(a) obtaining a myocardium sample from the individual;
(b) determining the level of a microRNA in the myocardium sample, wherein a difference in the level of the microRNA in the myocardium of an individual with heart disease from the level of the microRNA in a control individual who does not have heart disease indicates that the individual has heart disease;
(c) comparing the level of the microRNA in the myocardium sample to the level of the microRNA in the myocardium of a control individual who does not have heart disease; and,
(d) if the level of the microRNA in the myocardium sample of the individual is different from the level of the microRNA in the myocardium of the control individual diagnosing the individual as having heart disease.

3. (canceled)

4. The method of claim 2, wherein the microRNA is selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p, miR-499, miR-28, miR-222, miR-451, miR-422b, let-7g, miR-125a, miR-133a miR-133b, miR-15a, miR-16, miR-208, miR-30a-5p, miR-30b, miR-30c, miR-30d, miR-335, miR-195, let-7b, miR-27a, miR-27b, let-7c, miR-103, miR-23b, miR-24, miR-342, miR-23a, miR-145, miR-199a*, let-7e, miR-423*, miR-125b, miR-320, miR-93, miR-99b, miR-140*, miR-191, miR-15b, miR-181a, miR-100, and miR-214.

5. The method of claim 2, wherein the level of the microRNA in the myocardium of the individual is less than level of the microRNA in the myocardium of the control individual.

6. The method of claim 2, wherein the level of the microRNA in the myocardium of the individual is greater than level of the microRNA in the myocardium of the control individual.

7. The method of claim 5, wherein the microRNA is selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p, miR-499, miR-28, miR-222, miR-451, miR-422b, let-7g, miR-125a, miR-133a, miR-133b, miR-15a, miR-16, miR-208, miR-30a-5p, miR-30b, miR-30c, miR-30d, and miR-335.

8. The method of claim 6, wherein the microRNA is selected from the group consisting of: miR-195, let-7b, miR-27a, miR-27b,let-7c, miR-103, miR-23b, miR-24, miR-342, miR-23a, miR-145, miR-199a*, let-7e, miR-423*, miR-125b, miR-320, miR-93, miR-99b, miR-140*, miR-191, miR-15b, miR-181a, miR-100, and miR-214.

9. The method of claim 2, further comprising:
(a) determining the expression pattern of a set of microRNAs in a test myocardium sample obtained from the individual;
(b) comparing the expression pattern determined in (a) with one or more reference expression patterns, wherein each reference expression pattern is determined from the set of microRNAs in a reference myocardial sample obtained from an individual whose heart disease type is known; and
(c) categorizing the type of heart disease in the individual, as the known heart disease type associated with the reference expression pattern that most closely resembles the expression pattern determined in (a);
thereby determining the type of heart disease in the individual who has heart disease.

10-14. (canceled)

15. A method for modulating expression of genes associated with heart disease, comprising contacting a myocardial cell with an effective amount of a small-interfering nucleic acid capable of inhibiting, in myocardial cells, the expression of a gene product associated with heart disease, wherein the small-interfering nucleic acid comprises a sequence that is substantially similar to, or identical to, the sequence of an miRNA selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p,miR-499, miR-28, miR-222, miR-451, miR-422b, let-7g, miR-125a, miR-133a, miR-133b, miR-15a, miR-16, miR-208, miR-30a-5p, miR-30b, miR-30c, miR-30d, and miR-335.

16. The method of claim 15, wherein the gene product associated with heart disease is CX43, NFAT5, EDN1, CALM1, CALM2, or HDAC4.

17. (canceled)

18. The method of claim 15, wherein the small-interfering nucleic acid comprises the sequence provided in SEQ ID NO: 35.

19. The method of claim 15, wherein the heart disease is congestive heart failure, ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

20. A pharmaceutical formulation useful for modulating expression of genes associated with heart disease, comprising: (a) a small-interfering nucleic acid capable of inhibiting, in myocardial cells, the function of a gene product associated with heart disease, wherein the small-interfering nucleic acid comprises a sequence that is substantially similar to, or identical to, the sequence of an miRNA selected from the group consisting of: miR-10a, miR-19a, miR-19b, miR-101, miR-30e-5p, miR-126*, miR-374, miR-1, miR-20b, miR-20a, miR-26b, miR-126, miR-106a, miR-17-5p, miR-499, miR-28, miR-222, miR-451, miR-422b, let-7g, miR-125a, miR-133a, miR-133b, miR-15a, miR-16, miR-208, miR-30a-5p, miR-30b, miR-30c, miR-30d, and miR-335 and (b) a pharmaceutically acceptable carrier.

21. The pharmaceutical formulation of claim 20, wherein the small-interfering nucleic acid comprises the sequence provided in SEQ ID NO: 35.

22. The pharmaceutical formulation of claim 20, wherein the heart disease is congestive heart failure, ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

23-26. (canceled)

27. A pharmaceutical formulation useful for modulating expression of genes associated with heart disease, comprising: (a) a small-interfering nucleic acid capable of blocking, in a myocardial cell, the activity of an miRNA associated with heart disease, wherein the small-interfering nucleic acid comprises a sequence that is substantially complementary to, or complementary to, the sequence of the miRNA associated with heart disease, and wherein the miRNA associated with heart disease is selected from the group consisting of: miR-195, let-7b, miR-27a, miR-27b,let-7c, miR-103, miR-23b, miR-24, miR-342, miR-23a, miR-145, miR-199a*, let-7e, miR-423*, miR-125b, miR-320, miR-93, miR-99b, miR-140*, miR-191, miR-15b, miR-181a, miR-100, and miR-214 and (b) a pharmaceutically acceptable carrier.

28. The pharmaceutical formulation of claim 27, wherein the heart disease is congestive heart failure, ischemic cardiomyopathy, dilated cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis; aortic stenosis, aortic regurgitation, mitral stenosis, mitral regurgitation, mitral prolapse, pulmonary stenosis, pulmonary regurgitation, tricuspid stenosis, or tricuspid regurgitation.

29. (canceled)

30. The method of claim 2, wherein the myocardium sample is an RNA sample.

31. The method of claim 30, wherein determining comprises performing a bead-based assay, an array-based assay or a quantitative reverse transcription polymerase chain reaction assay to detect the microRNA in the RNA sample.

32. The method of claim 2, wherein determining comprises hybridizing a probe to the microRNA.

33. The method of claim 15, wherein the myocardial cell is in the individual.


SEQUENCE LISTING (47 sequences)

SEQ ID NO: 1 - DNA, 20 bp, Artificial Sequence
  Oligonucleotide
gggtcagaacccaacagaag

SEQ ID NO: 2 - DNA, 20 bp, Artificial Sequence
  Oligonucleotide
gcggatctcttcttcgctat

SEQ ID NO: 3 - DNA, 20 bp, Artificial Sequence
  Oligonucleotide
ggctgaactgcaggatatga

SEQ ID NO: 4 - DNA, 20 bp, Artificial Sequence
  Oligonucleotide
aatgcctcacggatttcttc

SEQ ID NO: 5 - DNA, 20 bp, Artificial Sequence
  Oligonucleotide
gcagaactgcaggacatgat

SEQ ID NO: 6 - DNA, 20 bp, Artificial Sequence
  Oligonucleotide
caaacacacggaatgcttct

SEQ ID NO: 7 - DNA, 20 bp, Artificial Sequence
  Oligonucleotide
cgagtcgagtggttgtctgt

SEQ ID NO: 8 - DNA, 20 bp, Artificial Sequence
  Oligonucleotide
ggttgttattgtcccatccc

SEQ ID NO: 9 - DNA, 20 bp, Artificial Sequence
  Oligonucleotide
tacctggtgctaacatccca

SEQ ID NO: 10 - DNA, 20 bp, Artificial Sequence
  Oligonucleotide
aagatcaccggcacattaca

SEQ ID NO: 11 - DNA, 20 bp, Artificial Sequence
  Oligonucleotide
gagacggccaggtcaattat

SEQ ID NO: 12 - DNA, 20 bp, Artificial Sequence
  Oligonucleotide
agaggagagcgcaagaagag

SEQ ID NO: 13 - DNA, 22 bp, Artificial Sequence
  Oligonucleotide
ccaagggagcatctttggactc

SEQ ID NO: 14 - DNA, 23 bp, Artificial Sequence
  Oligonucleotide
tgcttctaccacacacagcgaag

SEQ ID NO: 15 - DNA, 55 bp, Artificial Sequence
  Oligonucleotide
ctaggttcaaagaaattacagtttacgtccattccaagttgtaaatgctagtctt

SEQ ID NO: 16 - DNA, 55 bp, Artificial Sequence
  Oligonucleotide
agctaagactagcatttacaacttgaactggacgtaaactgtaatttctttgaac

SEQ ID NO: 17 - DNA, 25 bp, Artificial Sequence
  Oligonucleotide
tgtgcttctctccctcttttctcac

SEQ ID NO: 18 - DNA, 24 bp, Artificial Sequence
  Oligonucleotide
taactctgcgtggactatggacag

SEQ ID NO: 19 - DNA, 60 bp, Artificial Sequence
  Oligonucleotide
ctagtgcttatggcacaatttgcctcaaaatccattccaagttgtatatttgttttccaa

SEQ ID NO: 20 - DNA, 60 bp, Artificial Sequence
  Oligonucleotide
agctttggaaaacaaatatacaacttgaactggattttgaggcaaattgtgccataagca

SEQ ID NO: 21 - DNA, 32 bp, Artificial Sequence
  Oligonucleotide
ctagtgaattctacatacttctttacattcca

SEQ ID NO: 22 - DNA, 32 bp, Artificial Sequence
  Oligonucleotide
agcttggaatgtaaagaagtatgtagaattca

SEQ ID NO: 23 - DNA, 33 bp, Artificial Sequence
  Oligonucleotide
ctagtgaattcacagctggttgaaggggaccaa

SEQ ID NO: 24 - DNA, 33 bp, Artificial Sequence
  Oligonucleotide
agctttggtccccttcaaccagctgtgaattca

SEQ ID NO: 25 - DNA, 28 bp, Artificial Sequence
  Oligonucleotide
ccctcgagcactggatccattactcttc

SEQ ID NO: 26 - DNA, 28 bp, Artificial Sequence
  Oligonucleotide
ggtctagattggaatggggctgttagta

SEQ ID NO: 27 - RNA, 42 nt, Homo Sapiens
ucaaagaaauuacaguuuacguccauuccaaguuguaaaugc

SEQ ID NO: 28 - RNA, 41 nt, Mus sp.
ucaggaaaugauaauuuacguccauuccaaguuguaaaugc

SEQ ID NO: 29 - RNA, 41 nt, Rattus sp.; identical to SEQ ID NO: 28

SEQ ID NO: 30 - RNA, 41 nt, Canine sp.; identical to SEQ ID NO: 28

SEQ ID NO: 31 - RNA, 42 nt, Homo Sapiens
auggcacaauuugccucaaaauccauuccaaguuguauauuu

SEQ ID NO: 32 - RNA, 41 nt, Mus sp.
auggcacaauuugccucaaauccauuccaaguuguauauuu

SEQ ID NO: 33 - RNA, 41 nt, Rattus sp.; identical to SEQ ID NO: 32

SEQ ID NO: 34 - RNA, 41 nt, Canine sp.; identical to SEQ ID NO: 32

SEQ ID NO: 35 - RNA, 22 nt, Unknown
  miR-1
aauguaugaagaaauguaaggu

SEQ ID NO: 36 - RNA, 21 nt, Unknown
  hsa-miR-1-2
uggaauguaaagaaguaugua

SEQ ID NO: 37 - RNA, 21 nt, Unknown
  hsa-miR-1-1
uggaauguaaagaaguaugua

SEQ ID NO: 38 - RNA, 22 nt, Unknown
  hsa-miR-133a-1
uugguccccuucaaccagcugu

SEQ ID NO: 39 - RNA, 22 nt, Unknown
  hsa-miR-133a-2
uugguccccuucaaccagcugu

SEQ ID NO: 40 - RNA, 21 nt, Unknown
  hsa-miR-133b
uugguccccuucaaccagcua

SEQ ID NO: 41 - RNA, 22 nt, Unknown
  hsa-miR-30d
uguaaacauccccgacuggaag

SEQ ID NO: 42 - RNA, 20 nt, Unknown
  hsa-miR-30e-5p
uguaaacauccuugacugga

SEQ ID NO: 43 - RNA, 22 nt, Unknown
  hsa-miR-30a-5p
uguaaacauccucgacuggaag

SEQ ID NO: 44 - RNA, 22 nt, Unknown
  hsa-miR-30a-3p
cuuucagucggauguuugcagc

SEQ ID NO: 45 - RNA, 22 nt, Unknown
  hsa-miR-30b
uguaaacauccuacacucagcu

SEQ ID NO: 46 - RNA, 23 nt, Unknown
  hsa-miR-30c
uguaaacauccuacacucucagc

SEQ ID NO: 47 - RNA, 22 nt, Unknown
  hsa-miR-208
auaagacgagcaaaaagcuugu